SEQUENCE LISTING (36 sequences)

SEQ ID NO: 1 - protein, 1377 aa, Escherichia coli
MNRIYSLRYSAVARGFIAVSEFARKCVHKSVRRLCFPVLLLIPVLFSAGSLAGTVNNELGYQLFRDFAENKGMFRPGATNIAIYNKQGEFVGTLDKAAMPDFSAVDSEIGVATLINPQYIASVKHNGGYTNVSFGDGENRYNIVDRNNAPSLDFHAPRLDKLVTEVAPTAVTAQGAVAGAYLDKERYPVFYRLGSGTQYIKDSNGQLTKMGGAYSWLTGGTVGSLSSYQNGEMISTSSGLVFDYKLNGAMPIYGEAGDSGSPLFAFDTVQNKWVLVGVLTAGNGAGGRGNNWAVIPLDFIGQKFNEDNDAPVTFRTSEGGALEWSFNSSTGAGALTQGTTTYAMHGQQGNDLNAGKNLIFQGQNGQINLKDSVSQGAGSLTFRDNYTVTTSNGSTWTGAGIVVDNGVSVNWQVNGVKGDNLHKIGEGTLTVQGTGINEGGLKVGDGKVVLNQQADNKGQVQAFSSVNIASGRPTVVLTDERQVNPDTVSWGYRGGTLDVNGNSLTFHQLKAADYGAVLANNVDKRATITLDYALRADKVALNGWSESGKGTAGNLYKYNNPYTNTTDYFILKQSTYGYFPTDQSSNATWEFVGHSQGDAQKLVADRFNTAGYLFHGQLKGNLNVDNRLPEGVTGALVMDGAADISGTFTQENGRLTLQGHPVIHAYNTQSVADKLAASGDHSVLTQPTSFSQEDWENRSFTFDRLSLKNTDFGLGRNATLNTTIQADNSSVTLGDSRVFIDKNDGQGTAFTLEEGTSVATKDADKSVFNGTVNLDNQSVLNINDIFNGGIQANNSTVNISSDSAVLGNSTLTSTALNLNKGANALASQSFVSDGPVNISDATLSLNSRPDEVSHTLLPVYDYAGSWNLKGDDARLNVGPYSMLSGNINVQDKGTVTLGGEGELSPDLTLQNQMLYSLFNGYRNIWSGSLNAPDATVSMTDTQWSMNGNSTAGNMKLNRTIVGFNGGTSPFTTLTTDNLDAVQSAFVMRTDLNKADKLVINKSATGHDNSIWVNFLKKPSNKDTLDIPLVSAPEATADNLFRASTRVVGFSDVTPILSVRKEDGKKEWVLDGYQVARNDGQGKAAATFMHISYNNFITEVNNLNKRMGDLRDINGEAGTWVRLLNGSGSADGGFTDHYTLLQMGADRKHELGSMDLFTGVMATYTDTDASADLYSGKTKSWGGGFYASGLFRSGAYFDVIAKYIHNENKYDLNFAGAGKQNFRSHSLYAGAEVGYRYHLTDTTFVEPQAELVWGRLQGQTFNWNDSGMDVSMRRNSVNPLVGRTGVVSGKTFSGKDWSLTARAGLHYEFDLTDSADVHLKDAAGEHQINGRKDSRMLYGVGLNARFGDNTRLGLEVERSAFGKYNTDDAINANIRYSF

SEQ ID NO: 2 - protein, 1377 aa, Escherichia coli
MNRIYSLRYSAVARGFIAVSEFARKCVHKSVRRLCFPVLLLIPVLFSAGSLAGTVNNELGYQLFRDFAENKGMFRPGATNIAIYNKQGEFVGTLDKAAMPDFSAVDSEIGVATLINPQYIASVKHNGGYTNVSFGDGENRYNIVDRNNAPSLDFHAPRLDKLVTEVAPTAVTAQGAVAGAYLDKERYPVFYRLGSGTQYIKDSNGQLTKMGGAYSWLTGGTVGSLSSYQNGEMISTSSGLVFDYKLNGAMPIYGEAGDSGSPLFAFDTVQNKWVLVGVLTAGNGAGGRGNNWAVIPLDFIGQKFNEDNDAPVTFRTSEGGALEWSFNSSTGAGALTQGTTTYAMHGQQGNDLNAGKNLIFQGQNGQINLKDSVSQGAGSLTFRDNYTVTTSNGSTWTGAGIVVDNGVSVNWQVNGVKGDNLHKIGEGTLTVQGTGINEGGLKVGDGKVVLNQQADNKGQVQAFSSVNIASGRPTVVLTDERQVNPDTVSWGYRGGTLDVNGNSLTFHQLKAADYGAVLANNVDKRATITLDYALRADKVALNGWSESGKGTAGNLYKYNNPYTNTTDYFILKQSTYGYFPTDQSSNATWEFVGHSQGDAQKLVADRFNTAGYLFHGQLKGNLNVDNRLPEGVTGALVMDGAADISGTFTQENGRLTLQGHPVIHAYNTQSVADKLAASGDHSVLTQPTSFSQEDWENRSFTFDRLSLKNTDFGLGRNATLNTTIQADNSSVTLGDSRVFIDKNDGQGTAFTLEEGTSVATKDADKSVFNGTVNLDNQSVLNINDIFNGGIQANNSTVNISSDSAVLGNSTLTSTALNLNKGANALASQSFVSDGPVNISDATLSLNSRPDEVSHTLLPVYDYAGSWNLKGDDARLNVGPYSMLSGNINVQDKGTVTLGGEGELSPDLTLQNQMLYSLFNGYRNIWSGSLNAPDATVSMTDTQWSMNGNSTAGNMKLNRTIVGFNGGTSPFTTLTTDNLDAVQSAFVMRTDLNKADKLVINKSATGHDNSIWVNFLKKPSNKDTLDIPLVSAPEATADNLFRASTRVVGFSDVTPILSVRKEDGKKEWVLDGYQVARNDGQGKAAATFMHISYNNFITEVGSLNKRMGDLRDINGEAGTWVRLLNGSGSADGGFTDHYTLLQMGADRKHELGSMDLFTGVMATYTDTDASADLYSGKTKSWGGGFYASGLFRSGAYFDVIAKYIHNENKYDLNFAGAGKQNFRSHSLYAGAEVGYRYHLTDTTFVEPQAELVWGRLQGQTFNWNDSGMDVSMRRNSVNPLVGRTGVVSGKTFSGKDWSLTARAGLHYEFDLTDSADVHLKDAAGEHQINGRKDSRMLYGVGLNARFGDNTRLGLEVERSAFGKYNTDDAINANIRYSF

SEQ ID NO: 3 - protein, 1131 aa, Escherichia coli
MNRIYSLRYSAVARGFIAVSEFARKCVHKSVRRLCFPVLLLIPVLFSAGSLAGSSCGSGSGNDAPVTFRTSEGGALEWSFNSSTGAGALTQGTTTYAMHGQQGNDLNAGKNLIFQGQNGQINLKDSVSQGAGSLTFRDNYTVTTSNGSTWTGAGIVVDNGVSVNWQVNGVKGDNLHKIGEGTLTVQGTGINEGGLKVGDGKVVLNQQADNKGQVQAFSSVNIASGRPTVVLTDERQVNPDTVSWGYRGGTLDVNGNSLTFHQLKAADYGAVLANNVDKRATITLDYALRADKVALNGWSESGKGTAGNLYKYNNPYTNTTDYFILKQSTYGYFPTDQSSNATWEFVGHSQGDAQKLVADRFNTAGYLFHGQLKGNLNVDNRLPEGVTGALVMDGAADISGTFTQENGRLTLQGHPVIHAYNTQSVADKLAASGDHSVLTQPTSFSQEDWENRSFTFDRLSLKNTDFGLGRNATLNTTIQADNSSVTLGDSRVFIDKNDGQGTAFTLEEGTSVATKDADKSVFNGTVNLDNQSVLNINDIFNGGIQANNSTVNISSDSAVLGNSTLTSTALNLNKGANALASQSFVSDGPVNISDATLSLNSRPDEVSHTLLPVYDYAGSWNLKGDDARLNVGPYSMLSGNINVQDKGTVTLGGEGELSPDLTLQNQMLYSLFNGYRNIWSGSLNAPDATVSMTDTQWSMNGNSTAGNMKLNRTIVGFNGGTSPFTTLTTDNLDAVQSAFVMRTDLNKADKLVINKSATGHDNSIWVNFLKKPSNKDTLDIPLVSAPEATADNLFRASTRVVGFSDVTPILSVRKEDGKKEWVLDGYQVARNDGQGKAAATFMHISYNNFITEVGSLNKRMGDLRDINGEAGTWVRLLNGSGSADGGFTDHYTLLQMGADRKHELGSMDLFTGVMATYTDTDASADLYSGKTKSWGGGFYASGLFRSGAYFDVIAKYIHNENKYDLNFAGAGKQNFRSHSLYAGAEVGYRYHLTDTTFVEPQAELVWGRLQGQTFNWNDSGMDVSMRRNSVNPLVGRTGVVSGKTFSGKDWSLTARAGLHYEFDLTDSADVHLKDAAGEHQINGRKDSRMLYGVGLNARFGDNTRLGLEVERSAFGKYNTDDAINANIRYSF

SEQ ID NO: 4 - protein, 1289 aa, Escherichia coli
MNRIYSLRYSAVARGFIAVSEFARKCVHKSVRRLCFPVLLLIPVLFSAGSLAGSSEESPQVVEKSSLEKKYEEAKAKADTAKKDYETAKKKAEDAQKKYEDDQKRTEEKARKEAEASQKLNDVALVVQNAYKEYREVQNQRSKYKSDAEYQKKLTEVDSKIEKARKEQQDLQNKFNEVRAVVVPEPNGSGSGNDAPVTFRTSEGGALEWSFNSSTGAGALTQGTTTYAMHGQQGNDLNAGKNLIFQGQNGQINLKDSVSQGAGSLTFRDNYTVTTSNGSTWTGAGIVVDNGVSVNWQVNGVKGDNLHKIGEGTLTVQGTGINEGGLKVGDGKVVLNQQADNKGQVQAFSSVNIASGRPTVVLTDERQVNPDTVSWGYRGGTLDVNGNSLTFHQLKAADYGAVLANNVDKRATITLDYAGSGSSVRAVVVPEPNALAETKKKAEEAKAEEKVAKRKYDYATLKVALAKKEVEAKELEIEKLQYEISTLEQEVATAQHQVDNLKKLLAGADPDDGTGSGSGNTAGYLFHGQLKGNLNVDNRLPEGVTGALVMDGAADISGTFTQENGRLTLQGHPVIHAYNTQSVADKLAASGDHSVLTQPTSFSQEDWENRSFTFDRLSLKNTDFGLGRNATLNTTIQADNSSVTLGDSRVFIDKNDGQGTAFTLEEGTSVATKDADKSVFNGTVNLDNQSVLNINDIFNGGIQANNSTVNISSDSAVLGNSTLTSTALNLNKGANALASQSFVSDGPVNISDATLSLNSRPDEVSHTLLPVYDYAGSWNLKGDDARLNVGPYSMLSGNINVQDKGTVTLGGEGELSPDLTLQNQMLYSLFNGYRNIWSGSLNAPDATVSMTDTQWSMNGNSTAGNMKLNRTIVGFNGGTSPFTTLTTDNLDAVQSAFVMRTDLNKADKLVINKSATGHDNSIWVNFLKKPSNKDTLDIPLVSAPEATADNLFRASTRVVGFSDVTPILSVRKEDGKKEWVLDGYQVARNDGQGKAAATFMHISYNNFITEVGSLNKRMGDLRDINGEAGTWVRLLNGSGSADGGFTDHYTLLQMGADRKHELGSMDLFTGVMATYTDTDASADLYSGKTKSWGGGFYASGLFRSGAYFDVIAKYIHNENKYDLNFAGAGKQNFRSHSLYAGAEVGYRYHLTDTTFVEPQAELVWGRLQGQTFNWNDSGMDVSMRRNSVNPLVGRTGVVSGKTFSGKDWSLTARAGLHYEFDLTDSADVHLKDAAGEHQINGRKDSRMLYGVGLNARFGDNTRLGLEVERSAFGKYNTDDAINANIRYSF

SEQ ID NO: 5 - protein, 1202 aa, Escherichia coli
MNRIYSLRYSAVARGFIAVSEFARKCVHKSVRRLCFPVLLLIPVLFSAGSLAGSSCGSGSGNDAPVTFRTSEGGALEWSFNSSTGAGALTQGTTTYAMHGQQGNDLNAGKNLIFQGQNGQINLKDSVSQGAGSLTFRDNYTVTTSNGSTWTGAGIVVDNGVSVNWQVNGVKGDNLHKIGEGTLTVQGTGINEGGLKVGDGKVVLNQQADNKGQVQAFSSVNIASGRPTVVLTDERQVNPDTVSWGYRGGTLDVNGNSLTFHQLKAADYGAVLANNVDKRATITLDYALRADKVALNGWSESGKGTAGNLYKYNNPYTNTTDYFILKQSTYGYFPTDQSSNATWEFVGHSQGDAQKLVADRFNTAGYLFHGQLKGNLNVDNRLPEGVTGALVMDGAADISGTFTQENGRLTLQGHPVIHAYNTQSVADKLAASGDHSVLTQPTSFSQEDWENRSFTFDRLSLKNTDFGLGRNATLNTTIQADNSSVTLGDGSGSSELGPDGDEEETPAPAPQPEQPAPAPKPEQPAPAPKPEQPAPAPKPEQPAPAPKPEQPAPAPKPEQPAKPEKPAEEPTQPEKPATPKTGWKQGSGSGSVFNGTVNLDNQSVLNINDIFNGGIQANNSTVNISSDSAVLGNSTLTSTALNLNKGANALASQSFVSDGPVNISDATLSLNSRPDEVSHTLLPVYDYAGSWNLKGDDARLNVGPYSMLSGNINVQDKGTVTLGGEGELSPDLTLQNQMLYSLFNGYRNIWSGSLNAPDATVSMTDTQWSMNGNSTAGNMKLNRTIVGFNGGTSPFTTLTTDNLDAVQSAFVMRTDLNKADKLVINKSATGHDNSIWVNFLKKPSNKDTLDIPLVSAPEATADNLFRASTRVVGFSDVTPILSVRKEDGKKEWVLDGYQVARNDGQGKAAATFMHISYNNFITEVGSLNKRMGDLRDINGEAGTWVRLLNGSGSADGGFTDHYTLLQMGADRKHELGSMDLFTGVMATYTDTDASADLYSGKTKSWGGGFYASGLFRSGAYFDVIAKYIHNENKYDLNFAGAGKQNFRSHSLYAGAEVGYRYHLTDTTFVEPQAELVWGRLQGQTFNWNDSGMDVSMRRNSVNPLVGRTGVVSGKTFSGKDWSLTARAGLHYEFDLTDSADVHLKDAAGEHQINGRKDSRMLYGVGLNARFGDNTRLGLEVERSAFGKYNTDDAINANIRYSF

SEQ ID NO: 6 - protein, 1417 aa, Escherichia coli
MNRIYSLRYSAVARGFIAVSEFARKCVHKSVRRLCFPVLLLIPVLFSAGSLAGSSLAGADPDDGTEVIEAKLKKGEAELNAKQAELAKKQTELEKLLDSLDPEGKTQDELDKEAEEAELDKKADELQNKVADLEKEISNLEILLGGADPEDDTAALQNKLAAKKAELAKKQTELEKLLDSLDPEGKTQDELDKEAEEAELDKKADELQNKVADLEKEISNLEILLGGADSEDDTAALQNKLATKKAELEKTQKELDAALNELGPDGDEEETGSGSGNDAPVTFRTSEGGALEWSFNSSTGAGALTQGTTTYAMHGQQGNDLNAGKNLIFQGQNGQINLKDSVSQGAGSLTFRDNYTVTTSNGSTWTGAGIVVDNGVSVNWQVNGVKGDNLHKIGEGTLTVQGTGINEGGLKVGDGKVVLNQQADNKGQVQAFSSVNIASGRPTVVLTDERQVNPDTVSWGYRGGTLDVNGNSLTFHQLKAADYGAVLANNVDKRATITLDYALRADKVALNGWSESGKGTAGNLYKYNNPYTNTTDYFILKQSTYGYFPTDQSSNATWEFVGHSQGDAQKLVADRFNTAGYLFHGQLKGNLNVDNRLPEGVTGALVMDGAADISGTFTQENGRLTLQGHPVIHAYNTQSVADKLAASGDHSVLTQPTSFSQEDWENRSFTFDRLSLKNTDFGLGRNATLNTTIQADNSSVTLGDGSGSSELGPDGDEEETPAPAPQPEQPAPAPKPEQPAPAPKPEQPAPAPKPEQPAPAPKPEQPAPAPKPEQPAKPEKPAEEPTQPEKPATPKTGWKQGSGSGSVFNGTVNLDNQSVLNINDIFNGGIQANNSTVNISSDSAVLGNSTLTSTALNLNKGANALASQSFVSDGPVNISDATLSLNSRPDEVSHTLLPVYDYAGSWNLKGDDARLNVGPYSMLSGNINVQDKGTVTLGGEGELSPDLTLQNQMLYSLFNGYRNIWSGSLNAPDATVSMTDTQWSMNGNSTAGNMKLNRTIVGFNGGTSPFTTLTTDNLDAVQSAFVMRTDLNKADKLVINKSATGHDNSIWVNFLKKPSNKDTLDIPLVSAPEATADNLFRASTRVVGFSDVTPILSVRKEDGKKEWVLDGYQVARNDGQGKAAATFMHISYNNFITEVGSLNKRMGDLRDINGEAGTWVRLLNGSGSADGGFTDHYTLLQMGADRKHELGSMDLFTGVMATYTDTDASADLYSGKTKSWGGGFYASGLFRSGAYFDVIAKYIHNENKYDLNFAGAGKQNFRSHSLYAGAEVGYRYHLTDTTFVEPQAELVWGRLQGQTFNWNDSGMDVSMRRNSVNPLVGRTGVVSGKTFSGKDWSLTARAGLHYEFDLTDSADVHLKDAAGEHQINGRKDSRMLYGVGLNARFGDNTRLGLEVERSAFGKYNTDDAINANIRYSF

SEQ ID NO: 7 - protein, 1337 aa, Escherichia coli
MNRIYSLRYSAVARGFIAVSEFARKCVHKSVRRLCFPVLLLIPVLFSAGSLAGSSMANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHGSGSGNDAPVTFRTSEGGALEWSFNSSTGAGALTQGTTTYAMHGQQGNDLNAGKNLIFQGQNGQINLKDSVSQGAGSLTFRDNYTVTTSNGSTWTGAGIVVDNGVSVNWQVNGVKGDNLHKIGEGTLTVQGTGINEGGLKVGDGKVVLNQQADNKGQVQAFSSVNIASGRPTVVLTDERQVNPDTVSWGYRGGTLDVNGNSLTFHQLKAADYGAVLANNVDKRATITLDYAGSGSSAYRNGDLLLDHSGAYVAQYYITWNELSYDHQGKEVLTPKAWDRNGQDLTAHFTTSIPLKGNVRNLSVKIRECTGLAWEWWRTVYEKTDLPLVRKRTISIWGTTLYPQVEDKVENDGSGSGNTAGYLFHGQLKGNLNVDNRLPEGVTGALVMDGAADISGTFTQENGRLTLQGHPVIHAYNTQSVADKLAASGDHSVLTQPTSFSQEDWENRSFTFDRLSLKNTDFGLGRNATLNTTIQADNSSVTLGDSRVFIDKNDGQGTAFTLEEGTSVATKDADKSVFNGTVNLDNQSVLNINDIFNGGIQANNSTVNISSDSAVLGNSTLTSTALNLNKGANALASQSFVSDGPVNISDATLSLNSRPDEVSHTLLPVYDYAGSWNLKGDDARLNVGPYSMLSGNINVQDKGTVTLGGEGELSPDLTLQNQMLYSLFNGYRNIWSGSLNAPDATVSMTDTQWSMNGNSTAGNMKLNRTIVGFNGGTSPFTTLTTDNLDAVQSAFVMRTDLNKADKLVINKSATGHDNSIWVNFLKKPSNKDTLDIPLVSAPEATADNLFRASTRVVGFSDVTPILSVRKEDGKKEWVLDGYQVARNDGQGKAAATFMHISYNNFITEVGSLNKRMGDLRDINGEAGTWVRLLNGSGSADGGFTDHYTLLQMGADRKHELGSMDLFTGVMATYTDTDASADLYSGKTKSWGGGFYASGLFRSGAYFDVIAKYIHNENKYDLNFAGAGKQNFRSHSLYAGAEVGYRYHLTDTTFVEPQAELVWGRLQGQTFNWNDSGMDVSMRRNSVNPLVGRTGVVSGKTFSGKDWSLTARAGLHYEFDLTDSADVHLKDAAGEHQINGRKDSRMLYGVGLNARFGDNTRLGLEVERSAFGKYNTDDAINANIRYSF

SEQ ID NO: 8 - protein, 744 aa, Streptococcus pneumoniae
MNKKKMILTSLASVAILGAGFVTSQPTFVRAEESPQVVEKSSLEKKYEEAKAKADTAKKDYETAKKKAEDAQKKYEDDQKRTEEKARKEAEASQKLNDVALVVQNAYKEYREVQNQRSKYKSDAEYQKKLTEVDSKIEKARKEQQDLQNKFNEVRAVVVPEPNALAETKKKAEEAKAEEKVAKRKYDYATLKVALAKKEVEAKELEIEKLQYEISTLEQEVATAQHQVDNLKKLLAGADPDDGTEVIEAKLKKGEAELNAKQAELAKKQTELEKLLDSLDPEGKTQDELDKEAEEAELDKKADELQNKVADLEKEISNLEILLGGADPEDDTAALQNKLAAKKAELAKKQTELEKLLDSLDPEGKTQDELDKEAEEAELDKKADELQNKVADLEKEISNLEILLGGADSEDDTAALQNKLATKKAELEKTQKELDAALNELGPDGDEEETPAPAPQPEQPAPAPKPEQPAPAPKPEQPAPAPKPEQPAPAPKPEQPAPAPKPEQPAKPEKPAEEPTQPEKPATPKTGWKQENGMWYFYNTDGSMAIGWLQNNGSWYYLNANGAMATGWVKDGDTWYYLEASGAMKASQWFKVSDKWYYVNSNGAMATGWLQYNGSWYYLNANGDMATGWLQYNGSWYYLNANGDMATGWAKVNGSWYYLNANGAMATGWAKVNGSWYYLNANGSMATGWVKDGDTWYYLEASGAMKASQWFKVSDKWYYVNGLGALAVNTTVDGYKVNANGEWV

SEQ ID NO: 9 - protein, 262 aa, Streptococcus pneumoniae
EESPQVVEKSSLEKKYEEAKAKADTAKKDYETAKKKAEDAQKKYEDDQKRTEEKARKEAEASQKLNDVALVVQNAYKEYREVQNQRSKYKSDAEYQKKLTEVDSKIEKARKEQQDLQNKFNEVRAVVVPEPNALAETKKKAEEAKAEEKVAKRKYDYATLKVALAKKEVEAKELEIEKLQYEISTLEQEVATAQHQVDNLKKLLAGADPDDGTEVIEAKLKKGEAELNAKQAELAKKQTELEKLLDSLDPEGKTQDELDKEA

SEQ ID NO: 10 - protein, 132 aa, Streptococcus pneumoniae
EESPQVVEKSSLEKKYEEAKAKADTAKKDYETAKKKAEDAQKKYEDDQKRTEEKARKEAEASQKLNDVALVVQNAYKEYREVQNQRSKYKSDAEYQKKLTEVDSKIEKARKEQQDLQNKFNEVRAVVVPEPN

SEQ ID NO: 11 - protein, 91 aa, Streptococcus pneumoniae
VRAVVVPEPNALAETKKKAEEAKAEEKVAKRKYDYATLKVALAKKEVEAKELEIEKLQYEISTLEQEVATAQHQVDNLKKLLAGADPDDGT

SEQ ID NO: 12 - DNA, 4134 bp, Escherichia coli
atgaacagaatttattctcttcgctacagcgctgtggcccggggctttattgccgtatctgagtttgctaggaaatgtgttcataagtctgtcagacgtctgtgtttcccggttttattactgatcccggtactattctctgcaggaagtcttgcgggaacggtcaataatgaactcgggtatcagttatttcgtgattttgctgaaaataaggggatgttccgcccgggggcaacgaatatcgctatttataataagcagggagaatttgtcggtacgctggataaggcagctatgcctgatttcagtgctgtggattcggaaatcggtgtggcgacactgataaacccgcagtatatcgccagcgtgaaacataacgggggatatacaaacgttagctttggtgatggtgaaaaccgttacaatatcgtggaccggaataatgcgccgtcactggattttcatgccccccggctggataaactggtgacagaggttgcccctactgcggtgacggcgcagggggcagtggctggcgcatatctggataaggagcgctatcctgttttttatcgtctggggtctggtactcagtatattaaggacagtaacggacagctgacaaaaatgggaggtgcatattcctggctgaccggcgggactgtcggtagcctgtcatcctatcagaatggagaaatgattagcaccagttcaggtctggtttttgattacaaacttaatggtgcaatgcccatttatggcgaggccggtgacagcggttcgcctttatttgcttttgatactgttcagaataaatgggtgctggtcggtgttcttactgcggggaatggcgcggggggcaggggaaataactgggctgttattccactggattttatcgggcagaaatttaatgaagacaatgatgccccggtcacgttcagaacatcggaaggtggtgcactggagtggagctttaacagcagtaccggagctggtgcgctgacacagggaaccaccacatatgccatgcacgggcagcagggaaatgacctgaatgctggtaagaacctgatatttcaggggcagaatggtcagattaaccttaaggattcggtttctcagggggcgggttccctgacgttccgtgataattacacagtaacaacctctaacggaagtacctggaccggtgccggtattgttgtggacaacggggtgtccgtaaactggcaggttaatggtgttaagggcgataacctgcataaaattggtgaaggtacgctgacggtacagggtacaggtattaatgaaggtggcctgaaggtcggggacggaaaggttgtactgaaccagcaggcggacaataaaggacaggtgcaggcgttcagcagtgttaatattgccagtggccggccgaccgtggtactgactgatgagcggcaggtaaatccggataccgtctcatggggatatcgtgggggcacactggatgttaatggtaacagtctgacgtttcatcagttgaaggcggcagattatggtgccgtgctggcgaataacgttgataaacgggccactatcacgctggactatgccctgcgggctgacaaagtagcactgaatggctggtcggaatcaggtaaaggaactgccggaaatttatataaatacaataacccgtacacaaatacgacggattacttcatcctgaagcagagcacctatggttatttccccacggaccagagcagcaacgccacctgggagtttgtggggcacagtcagggggatgcacagaaactggtagctgaccgtttcaatactgcagggtatctgtttcacggacaactgaaaggcaatctgaatgtggacaatcgcctgcctgaaggcgttaccggtgctctggtgatggacggagctgcggatatctccggtacattcacccaggaaaacgggcgtctgacgctgcaggggcatccggttatccatgcatacaatactcagtctgtggctgacaaactggctgccagtggagaccattcggttctgactcagcctacgtcattcagtcaggaggactgggagaaccgcagttttacctttgacaggctgtcactgaagaacactgattttggtcttggtcgcaatgccacactgaacacaaccatccaggcagataactccagcgtcacgctgggcgacagccgggtatttatcgacaaaaacgatggccagggaacagcctttacccttgaagaaggcacatctgttgcaactaaagatgcagataaaagtgtcttcaacggcaccgtcaacctggataatcagtcagtgctgaatatcaatgatatattcaatggcggaatacaggcgaacaacagtaccgtgaatatctcctcagacagtgccgttctggggaactcaacactgaccagtaccgccctgaatctgaacaagggagcaaatgctctggccagtcagagttttgtttctgacggtccagtgaatatttctgatgccaccctgagtctgaacagccgtcctgatgaggtatctcacacacttttacctgtatacgattatgccggttcatggaacctgaagggagacgatgcccgcctgaacgtggggccgtacagtatgttgtcaggtaatatcaatgttcaggataaagggactgtcaccctcggaggggaaggggaactgagtcctgacctgactcttcagaatcagatgttgtacagcctgtttaacgggtaccgcaatatctggagcgggagcctgaatgcaccggatgccaccgtcagcatgacagacacccagtggtcgatgaacggaaactccacggcaggaaatatgaaacttaaccggacaatagtcggttttaacgggggaacatcaccgttcacgacactgacaacagataatctggacgcggttcagtcagcatttgtcatgcgtacagaccttaacaaggcagacaaactggtgataaacaagtcggcaacaggtcatgacaacagcatctgggttaacttcctgaaaaaaccttctaacaaggacacgcttgatattccactggtcagcgcacctgaagcgacagctgataatctgttcagggcatcaacacgggttgtgggattcagtgatgtcacccccatccttagtgtcagaaaagaggacgggaaaaaagagtgggtcctcgatggttaccaggttgcacgtaacgacggccagggtaaggctgccgccacattcatgcacatcagctataacaacttcatcactgaagttaacaacctgaacaaacgcatgggcgatttgagggatattaatggcgaagccggtacgtgggtgcgtctgctgaacggttccggctctgctgatggcggtttcactgaccactataccctgctgcagatgggggctgaccgtaagcacgaactgggaagtatggacctgtttaccggcgtgatggccacctacactgacacagatgcgtcagcagacctgtacagcggtaaaacaaaatcatggggtggtggtttctatgccagtggtctgttccggtccggcgcttactttgatgtgattgccaaatatattcacaatgaaaacaaatatgacctgaactttgccggagctggtaaacagaacttccgcagccattcactgtatgcaggtgcagaagtcggataccgttatcatctgacagatacgacgtttgttgaacctcaggcggaactggtctggggaagactgcagggccaaacatttaactggaacgacagtggaatggatgtctcaatgcgtcgtaacagcgttaatcctctggtaggcagaaccggcgttgtttccggtaaaaccttcagtggtaaggactggagtctgacagcccgtgccggcctgcattatgagttcgatctgacggacagtgctgacgttcatctgaaggatgcagcgggagaacatcagattaatggcagaaaagacagtcgtatgctttacggtgtggggttaaatgcccggtttggcgacaatacgcgtctggggctggaagttgaacgctctgcatttggtaaatacaacacagatgatgcgataaacgctaatattcgttattcattctga

SEQ ID NO: 13 - DNA, 4134 bp, Escherichia coli
atgaacagaatttattctcttcgctacagcgctgtggcccggggctttattgccgtatctgagtttgctaggaaatgtgttcataagtctgtcagacgtctgtgtttcccggttttattactgatcccggtactattctctgcaggaagtcttgcgggaacggtcaataatgaactcgggtatcagttatttcgtgattttgctgaaaataaggggatgttccgcccgggggcaacgaatatcgctatttataataagcagggagaatttgtcggtacgctggataaggcagctatgcctgatttcagtgctgtggattcggaaatcggtgtggcgacactgataaacccgcagtatatcgccagcgtgaaacataacgggggatatacaaacgttagctttggtgatggtgaaaaccgttacaatatcgtggaccggaataatgcgccgtcactggattttcatgccccccggctggataaactggtgacagaggttgcccctactgcggtgacggcgcagggggcagtggctggcgcatatctggataaggagcgctatcctgttttttatcgtctggggtctggtactcagtatattaaggacagtaacggacagctgacaaaaatgggaggtgcatattcctggctgaccggcgggactgtcggtagcctgtcatcctatcagaatggagaaatgattagcaccagttcaggtctggtttttgattacaaacttaatggtgcaatgcccatttatggcgaggccggtgacagcggttcgcctttatttgcttttgatactgttcagaataaatgggtgctggtcggtgttcttactgcggggaatggcgcggggggcaggggaaataactgggctgttattccactggattttatcgggcagaaatttaatgaagacaatgatgccccggtcacgttcagaacatcggaaggtggtgcactggagtggagctttaacagcagtaccggagctggtgcgctgacacagggaaccaccacatatgccatgcacgggcagcagggaaatgacctgaatgctggtaagaacctgatatttcaggggcagaatggtcagattaaccttaaggattcggtttctcagggggcgggttccctgacgttccgtgataattacacagtaacaacctctaacggaagtacctggaccggtgccggtattgttgtggacaacggggtgtccgtaaactggcaggttaatggtgttaagggcgataacctgcataaaattggtgaaggtacgctgacggtacagggtacaggtattaatgaaggtggcctgaaggtcggggacggaaaggttgtactgaaccagcaggcggacaataaaggacaggtgcaggcgttcagcagtgttaatattgccagtggccggccgaccgtggtactgactgatgagcggcaggtaaatccggataccgtctcatggggatatcgtgggggcacactggatgttaatggtaacagtctgacgtttcatcagttgaaggcggcagattatggtgccgtgctggcgaataacgttgataaacgggccactatcacgctggactatgccctgcgggctgacaaagtagcactgaatggctggtcggaatcaggtaaaggaactgccggaaatttatataaatacaataacccgtacacaaatacgacggattacttcatcctgaagcagagcacctatggttatttccccacggaccagagcagcaacgccacctgggagtttgtggggcacagtcagggggatgcacagaaactggtagctgaccgtttcaatactgcagggtatctgtttcacggacaactgaaaggcaatctgaatgtggacaatcgcctgcctgaaggcgttaccggtgctctggtgatggacggagctgcggatatctccggtacattcacccaggaaaacgggcgtctgacgctgcaggggcatccggttatccatgcatacaatactcagtctgtggctgacaaactggctgccagtggagaccattcggttctgactcagcctacgtcattcagtcaggaggactgggagaaccgcagttttacctttgacaggctgtcactgaagaacactgattttggtcttggtcgcaatgccacactgaacacaaccatccaggcagataactccagcgtcacgctgggcgacagccgggtatttatcgacaaaaacgatggccagggaacagcctttacccttgaagaaggcacatctgttgcaactaaagatgcagataaaagtgtcttcaacggcaccgtcaacctggataatcagtcagtgctgaatatcaatgatatattcaatggcggaatacaggcgaacaacagtaccgtgaatatctcctcagacagtgccgttctggggaactcaacactgaccagtaccgccctgaatctgaacaagggagcaaatgctctggccagtcagagttttgtttctgacggtccagtgaatatttctgatgccaccctgagtctgaacagccgtcctgatgaggtatctcacacacttttacctgtatacgattatgccggttcatggaacctgaagggagacgatgcccgcctgaacgtggggccgtacagtatgttgtcaggtaatatcaatgttcaggataaagggactgtcaccctcggaggggaaggggaactgagtcctgacctgactcttcagaatcagatgttgtacagcctgtttaacgggtaccgcaatatctggagcgggagcctgaatgcaccggatgccaccgtcagcatgacagacacccagtggtcgatgaacggaaactccacggcaggaaatatgaaacttaaccggacaatagtcggttttaacgggggaacatcaccgttcacgacactgacaacagataatctggacgcggttcagtcagcatttgtcatgcgtacagaccttaacaaggcagacaaactggtgataaacaagtcggcaacaggtcatgacaacagcatctgggttaacttcctgaaaaaaccttctaacaaggacacgcttgatattccactggtcagcgcacctgaagcgacagctgataatctgttcagggcatcaacacgggttgtgggattcagtgatgtcacccccatccttagtgtcagaaaagaggacgggaaaaaagagtgggtcctcgatggttaccaggttgcacgtaacgacggccagggtaaggctgccgccacattcatgcacatcagctataacaacttcatcactgaagttggttccctgaacaaacgcatgggcgatttgagggatattaatggcgaagccggtacgtgggtgcgtctgctgaacggttccggctctgctgatggcggtttcactgaccactataccctgctgcagatgggggctgaccgtaagcacgaactgggaagtatggacctgtttaccggcgtgatggccacctacactgacacagatgcgtcagcagacctgtacagcggtaaaacaaaatcatggggtggtggtttctatgccagtggtctgttccggtccggcgcttactttgatgtgattgccaaatatattcacaatgaaaacaaatatgacctgaactttgccggagctggtaaacagaacttccgcagccattcactgtatgcaggtgcagaagtcggataccgttatcatctgacagatacgacgtttgttgaacctcaggcggaactggtctggggaagactgcagggccaaacatttaactggaacgacagtggaatggatgtctcaatgcgtcgtaacagcgttaatcctctggtaggcagaaccggcgttgtttccggtaaaaccttcagtggtaaggactggagtctgacagcccgtgccggcctgcattatgagttcgatctgacggacagtgctgacgttcatctgaaggatgcagcgggagaacatcagattaatggcagaaaagacagtcgtatgctttacggtgtggggttaaatgcccggtttggcgacaatacgcgtctggggctggaagttgaacgctctgcatttggtaaatacaacacagatgatgcgataaacgctaatattcgttattcattctga

SEQ ID NO: 14 - DNA, 3396 bp, Escherichia coli
atgaacagaatttattctcttcgctacagcgctgtggcccggggctttattgccgtatctgagtttgctaggaaatgtgttcataagtctgtcagacgtctgtgtttcccggttttattactgatcccggtactattctctgcaggaagtcttgcggggagctcctgcggatccggcagcggtaatgatgccccggtcacgttcagaacatcggaaggtggtgcactggagtggagctttaacagcagtaccggagctggtgcgctgacacagggaaccaccacatatgccatgcacgggcagcagggaaatgacctgaatgctggtaagaacctgatatttcaggggcagaatggtcagattaaccttaaggattcggtttctcagggggcgggttccctgacgttccgtgataattacacagtaacaacctctaacggaagtacctggaccggtgccggtattgttgtggacaacggggtgtccgtaaactggcaggttaatggtgttaagggcgataacctgcataaaattggtgaaggtacgctgacggtacagggtacaggtattaatgaaggtggcctgaaggtcggggacggaaaggttgtactgaaccagcaggcggacaataaaggacaggtgcaggcgttcagcagtgttaatattgccagtggccggccgaccgtggtactgactgatgagcggcaggtaaatccggataccgtctcatggggatatcgtgggggcacactggatgttaatggtaacagtctgacgtttcatcagttgaaggcggcagattatggtgccgtgctggcgaataacgttgataaacgggccactatcacgctggactatgccctgcgggctgacaaagtagcactgaatggctggtcggaatcaggtaaaggaactgccggaaatttatataaatacaataacccgtacacaaatacgacggattacttcatcctgaagcagagcacctatggttatttccccacggaccagagcagcaacgccacctgggagtttgtggggcacagtcagggggatgcacagaaactggtagctgaccgtttcaatactgcagggtatctgtttcacggacaactgaaaggcaatctgaatgtggacaatcgcctgcctgaaggcgttaccggtgctctggtgatggacggagctgcggatatctccggtacattcacccaggaaaacgggcgtctgacgctgcaggggcatccggttatccatgcatacaatactcagtctgtggctgacaaactggctgccagtggagaccattcggttctgactcagcctacgtcattcagtcaggaggactgggagaaccgcagttttacctttgacaggctgtcactgaagaacactgattttggtcttggtcgcaatgccacactgaacacaaccatccaggcagataactccagcgtcacgctgggcgacagccgggtatttatcgacaaaaacgatggccagggaacagcctttacccttgaagaaggcacatctgttgcaactaaagatgcagataaaagtgtcttcaacggcaccgtcaacctggataatcagtcagtgctgaatatcaatgatatattcaatggcggaatacaggcgaacaacagtaccgtgaatatctcctcagacagtgccgttctggggaactcaacactgaccagtaccgccctgaatctgaacaagggagcaaatgctctggccagtcagagttttgtttctgacggtccagtgaatatttctgatgccaccctgagtctgaacagccgtcctgatgaggtatctcacacacttttacctgtatacgattatgccggttcatggaacctgaagggagacgatgcccgcctgaacgtggggccgtacagtatgttgtcaggtaatatcaatgttcaggataaagggactgtcaccctcggaggggaaggggaactgagtcctgacctgactcttcagaatcagatgttgtacagcctgtttaacgggtaccgcaatatctggagcgggagcctgaatgcaccggatgccaccgtcagcatgacagacacccagtggtcgatgaacggaaactccacggcaggaaatatgaaacttaaccggacaatagtcggttttaacgggggaacatcaccgttcacgacactgacaacagataatctggacgcggttcagtcagcatttgtcatgcgtacagaccttaacaaggcagacaaactggtgataaacaagtcggcaacaggtcatgacaacagcatctgggttaacttcctgaaaaaaccttctaacaaggacacgcttgatattccactggtcagcgcacctgaagcgacagctgataatctgttcagggcatcaacacgggttgtgggattcagtgatgtcacccccatccttagtgtcagaaaagaggacgggaaaaaagagtgggtcctcgatggttaccaggttgcacgtaacgacggccagggtaaggctgccgccacattcatgcacatcagctataacaacttcatcactgaagttggttccctgaacaaacgcatgggcgatttgagggatattaatggcgaagccggtacgtgggtgcgtctgctgaacggttccggctctgctgatggcggtttcactgaccactataccctgctgcagatgggggctgaccgtaagcacgaactgggaagtatggacctgtttaccggcgtgatggccacctacactgacacagatgcgtcagcagacctgtacagcggtaaaacaaaatcatggggtggtggtttctatgccagtggtctgttccggtccggcgcttactttgatgtgattgccaaatatattcacaatgaaaacaaatatgacctgaactttgccggagctggtaaacagaacttccgcagccattcactgtatgcaggtgcagaagtcggataccgttatcatctgacagatacgacgtttgttgaacctcaggcggaactggtctggggaagactgcagggccaaacatttaactggaacgacagtggaatggatgtctcaatgcgtcgtaacagcgttaatcctctggtaggcagaaccggcgttgtttccggtaaaaccttcagtggtaaggactggagtctgacagcccgtgccggcctgcattatgagttcgatctgacggacagtgctgacgttcatctgaaggatgcagcgggagaacatcagattaatggcagaaaagacagtcgtatgctttacggtgtggggttaaatgcccggtttggcgacaatacgcgtctggggctggaagttgaacgctctgcatttggtaaatacaacacagatgatgcgataaacgctaatattcgttattcattctga

SEQ ID NO: 15 - DNA, 3870 bp, Escherichia coli
atgaacagaatttattctcttcgctacagcgctgtggcccggggctttattgccgtatctgagtttgctaggaaatgtgttcataagtctgtcagacgtctgtgtttcccggttttattactgatcccggtactattctctgcaggaagtcttgcggggagctccgaagaatctccacaagttgtcgaaaaatcttcattagagaagaaatatgaggaagcaaaagcaaaagctgatactgccaagaaagattacgaaacggctaaaaagaaagcagaagacgctcagaaaaagtatgaagatgatcagaagagaactgaggagaaagctcgaaaagaagcagaagcatctcaaaaattgaatgatgtggcgcttgttgttcaaaatgcatataaagagtaccgagaagttcaaaatcaacgtagtaaatataaatctgacgctgaatatcagaaaaaattaacagaggtcgactctaaaatagagaaggctaggaaagagcaacaggacttgcaaaataaatttaatgaagtaagagcagttgtagttcctgaaccaaatggatccggcagcggtaatgatgccccggtcacgttcagaacatcggaaggtggtgcactggagtggagctttaacagcagtaccggagctggtgcgctgacacagggaaccaccacatatgccatgcacgggcagcagggaaatgacctgaatgctggtaagaacctgatatttcaggggcagaatggtcagattaaccttaaggattcggtttctcagggggcgggttccctgacgttccgtgataattacacagtaacaacctctaacggaagtacctggaccggtgccggtattgttgtggacaacggggtgtccgtaaactggcaggttaatggtgttaagggcgataacctgcataaaattggtgaaggtacgctgacggtacagggtacaggtattaatgaaggtggcctgaaggtcggggacggaaaggttgtactgaaccagcaggcggacaataaaggacaggtgcaggcgttcagcagtgttaatattgccagtggccggccgaccgtggtactgactgatgagcggcaggtaaatccggataccgtctcatggggatatcgtgggggcacactggatgttaatggtaacagtctgacgtttcatcagttgaaggcggcagattatggtgccgtgctggcgaataacgttgataaacgggccactatcacgctggactatgccggttctgggagctccgtaagagcagttgtagttcctgaaccaaatgcgttggctgagactaagaaaaaagcagaagaagctaaagcagaagaaaaagtagctaagagaaaatatgattatgcaactctaaaggtagcactagcgaagaaagaagtagaggctaaggaacttgaaattgaaaaacttcaatatgaaatttctactttggaacaagaagttgctactgctcaacatcaagtagataatttgaaaaaacttcttgctggtgcagatcctgatgatggcacaggatccggcagcggtaatactgcagggtatctgtttcacggacaactgaaaggcaatctgaatgtggacaatcgcctgcctgaaggcgttaccggtgctctggtgatggacggagctgcggatatctccggtacattcacccaggaaaacgggcgtctgacgctgcaggggcatccggttatccatgcatacaatactcagtctgtggctgacaaactggctgccagtggagaccattcggttctgactcagcctacgtcattcagtcaggaggactgggagaaccgcagttttacctttgacaggctgtcactgaagaacactgattttggtcttggtcgcaatgccacactgaacacaaccatccaggcagataactccagcgtcacgctgggcgacagccgggtatttatcgacaaaaacgatggccagggaacagcctttacccttgaagaaggcacatctgttgcaactaaagatgcagataaaagtgtcttcaacggcaccgtcaacctggataatcagtcagtgctgaatatcaatgatatattcaatggcggaatacaggcgaacaacagtaccgtgaatatctcctcagacagtgccgttctggggaactcaacactgaccagtaccgccctgaatctgaacaagggagcaaatgctctggccagtcagagttttgtttctgacggtccagtgaatatttctgatgccaccctgagtctgaacagccgtcctgatgaggtatctcacacacttttacctgtatacgattatgccggttcatggaacctgaagggagacgatgcccgcctgaacgtggggccgtacagtatgttgtcaggtaatatcaatgttcaggataaagggactgtcaccctcggaggggaaggggaactgagtcctgacctgactcttcagaatcagatgttgtacagcctgtttaacgggtaccgcaatatctggagcgggagcctgaatgcaccggatgccaccgtcagcatgacagacacccagtggtcgatgaacggaaactccacggcaggaaatatgaaacttaaccggacaatagtcggttttaacgggggaacatcaccgttcacgacactgacaacagataatctggacgcggttcagtcagcatttgtcatgcgtacagaccttaacaaggcagacaaactggtgataaacaagtcggcaacaggtcatgacaacagcatctgggttaacttcctgaaaaaaccttctaacaaggacacgcttgatattccactggtcagcgcacctgaagcgacagctgataatctgttcagggcatcaacacgggttgtgggattcagtgatgtcacccccatccttagtgtcagaaaagaggacgggaaaaaagagtgggtcctcgatggttaccaggttgcacgtaacgacggccagggtaaggctgccgccacattcatgcacatcagctataacaacttcatcactgaagttggttccctgaacaaacgcatgggcgatttgagggatattaatggcgaagccggtacgtgggtgcgtctgctgaacggttccggctctgctgatggcggtttcactgaccactataccctgctgcagatgggggctgaccgtaagcacgaactgggaagtatggacctgtttaccggcgtgatggccacctacactgacacagatgcgtcagcagacctgtacagcggtaaaacaaaatcatggggtggtggtttctatgccagtggtctgttccggtccggcgcttactttgatgtgattgccaaatatattcacaatgaaaacaaatatgacctgaactttgccggagctggtaaacagaacttccgcagccattcactgtatgcaggtgcagaagtcggataccgttatcatctgacagatacgacgtttgttgaacctcaggcggaactggtctggggaagactgcagggccaaacatttaactggaacgacagtggaatggatgtctcaatgcgtcgtaacagcgttaatcctctggtaggcagaaccggcgttgtttccggtaaaaccttcagtggtaaggactggagtctgacagcccgtgccggcctgcattatgagttcgatctgacggacagtgctgacgttcatctgaaggatgcagcgggagaacatcagattaatggcagaaaagacagtcgtatgctttacggtgtggggttaaatgcccggtttggcgacaatacgcgtctggggctggaagttgaacgctctgcatttggtaaatacaacacagatgatgcgataaacgctaatattcgttattcattctga

SEQ ID NO: 16 - DNA, 3609 bp, Escherichia coli
atgaacagaatttattctcttcgctacagcgctgtggcccggggctttattgccgtatctgagtttgctaggaaatgtgttcataagtctgtcagacgtctgtgtttcccggttttattactgatcccggtactattctctgcaggaagtcttgcggggagctcctgcggatccggcagcggtaatgatgccccggtcacgttcagaacatcggaaggtggtgcactggagtggagctttaacagcagtaccggagctggtgcgctgacacagggaaccaccacatatgccatgcacgggcagcagggaaatgacctgaatgctggtaagaacctgatatttcaggggcagaatggtcagattaaccttaaggattcggtttctcagggggcgggttccctgacgttccgtgataattacacagtaacaacctctaacggaagtacctggaccggtgccggtattgttgtggacaacggggtgtccgtaaactggcaggttaatggtgttaagggcgataacctgcataaaattggtgaaggtacgctgacggtacagggtacaggtattaatgaaggtggcctgaaggtcggggacggaaaggttgtactgaaccagcaggcggacaataaaggacaggtgcaggcgttcagcagtgttaatattgccagtggccggccgaccgtggtactgactgatgagcggcaggtaaatccggataccgtctcatggggatatcgtgggggcacactggatgttaatggtaacagtctgacgtttcatcagttgaaggcggcagattatggtgccgtgctggcgaataacgttgataaacgggccactatcacgctggactatgccctgcgggctgacaaagtagcactgaatggctggtcggaatcaggtaaaggaactgccggaaatttatataaatacaataacccgtacacaaatacgacggattacttcatcctgaagcagagcacctatggttatttccccacggaccagagcagcaacgccacctgggagtttgtggggcacagtcagggggatgcacagaaactggtagctgaccgtttcaatactgcagggtatctgtttcacggacaactgaaaggcaatctgaatgtggacaatcgcctgcctgaaggcgttaccggtgctctggtgatggacggagctgcggatatctccggtacattcacccaggaaaacgggcgtctgacgctgcaggggcatccggttatccatgcatacaatactcagtctgtggctgacaaactggctgccagtggagaccattcggttctgactcagcctacgtcattcagtcaggaggactgggagaaccgcagttttacctttgacaggctgtcactgaagaacactgattttggtcttggtcgcaatgccacactgaacacaaccatccaggcagataactccagcgtcacgctgggcgacgggagcgggagctccgagttaggccctgatggagatgaagaagaaactccagcgccggctcctcaaccagagcaaccagctcctgcaccaaaaccagagcaaccagctccagctccaaaaccagagcaaccagctcctgcaccaaaaccagagcaaccagctccagctccaaaaccagagcaaccagctccagctccaaaaccagagcaaccagctaagccggagaaaccagctgaagagcctactcaaccagaaaaaccagccactccaaaaacaggctggaaacaaggatccggcagcggtagtgtcttcaacggcaccgtcaacctggataatcagtcagtgctgaatatcaatgatatattcaatggcggaatacaggcgaacaacagtaccgtgaatatctcctcagacagtgccgttctggggaactcaacactgaccagtaccgccctgaatctgaacaagggagcaaatgctctggccagtcagagttttgtttctgacggtccagtgaatatttctgatgccaccctgagtctgaacagccgtcctgatgaggtatctcacacacttttacctgtatacgattatgccggttcatggaacctgaagggagacgatgcccgcctgaacgtggggccgtacagtatgttgtcaggtaatatcaatgttcaggataaagggactgtcaccctcggaggggaaggggaactgagtcctgacctgactcttcagaatcagatgttgtacagcctgtttaacgggtaccgcaatatctggagcgggagcctgaatgcaccggatgccaccgtcagcatgacagacacccagtggtcgatgaacggaaactccacggcaggaaatatgaaacttaaccggacaatagtcggttttaacgggggaacatcaccgttcacgacactgacaacagataatctggacgcggttcagtcagcatttgtcatgcgtacagaccttaacaaggcagacaaactggtgataaacaagtcggcaacaggtcatgacaacagcatctgggttaacttcctgaaaaaaccttctaacaaggacacgcttgatattccactggtcagcgcacctgaagcgacagctgataatctgttcagggcatcaacacgggttgtgggattcagtgatgtcacccccatccttagtgtcagaaaagaggacgggaaaaaagagtgggtcctcgatggttaccaggttgcacgtaacgacggccagggtaaggctgccgccacattcatgcacatcagctataacaacttcatcactgaagttggttccctgaacaaacgcatgggcgatttgagggatattaatggcgaagccggtacgtgggtgcgtctgctgaacggttccggctctgctgatggcggtttcactgaccactataccctgctgcagatgggggctgaccgtaagcacgaactgggaagtatggacctgtttaccggcgtgatggccacctacactgacacagatgcgtcagcagacctgtacagcggtaaaacaaaatcatggggtggtggtttctatgccagtggtctgttccggtccggcgcttactttgatgtgattgccaaatatattcacaatgaaaacaaatatgacctgaactttgccggagctggtaaacagaacttccgcagccattcactgtatgcaggtgcagaagtcggataccgttatcatctgacagatacgacgtttgttgaacctcaggcggaactggtctggggaagactgcagggccaaacatttaactggaacgacagtggaatggatgtctcaatgcgtcgtaacagcgttaatcctctggtaggcagaaccggcgttgtttccggtaaaaccttcagtggtaaggactggagtctgacagcccgtgccggcctgcattatgagttcgatctgacggacagtgctgacgttcatctgaaggatgcagcgggagaacatcagattaatggcagaaaagacagtcgtatgctttacggtgtggggttaaatgcccggtttggcgacaatacgcgtctggggctggaagttgaacgctctgcatttggtaaatacaacacagatgatgcgataaacgctaatattcgttattcattctga

SEQ ID NO: 17 - DNA, 4254 bp, Escherichia coli
atgaacagaatttattctcttcgctacagcgctgtggcccggggctttattgccgtatctgagtttgctaggaaatgtgttcataagtctgtcagacgtctgtgtttcccggttttattactgatcccggtactattctctgcaggaagtcttgcggggagctccctggctggtgcagatccggatgatggcaccgaagttattgaagcaaaactgaaaaaaggtgaagccgaactgaatgcaaaacaggcagaactggcaaaaaaacagaccgagctggaaaaactgctggatagcctggacccggaaggtaaaacccaggatgaactggataaagaagcagaagaagcggagctggacaaaaaagcagatgaactgcagaataaagttgcggatctggaaaaagaaatcagcaacctggaaattctgctgggtggtgccgatcctgaagatgataccgcagcactgcagaacaaactggcagcaaaaaaagcggaactggccaaaaaacaaacggaactggaaaaactgctggactcactggaccctgagggcaaaacacaagacgagctggataaagaggccgaggaagcagaactggataaaaaagccgacgagctgcaaaacaaagtggccgacctggaaaaagagatttcaaatctggaaatcctgctgggaggcgcagatagcgaggatgacacagctgcactgcaaaataaactggccacgaaaaaagcagagctggaaaaaacccagaaagaactggacgcagcactgaatgaactgggtccggatggtgatgaagaagaaaccggatccggcagcggtaatgatgccccggtcacgttcagaacatcggaaggtggtgcactggagtggagctttaacagcagtaccggagctggtgcgctgacacagggaaccaccacatatgccatgcacgggcagcagggaaatgacctgaatgctggtaagaacctgatatttcaggggcagaatggtcagattaaccttaaggattcggtttctcagggggcgggttccctgacgttccgtgataattacacagtaacaacctctaacggaagtacctggaccggtgccggtattgttgtggacaacggggtgtccgtaaactggcaggttaatggtgttaagggcgataacctgcataaaattggtgaaggtacgctgacggtacagggtacaggtattaatgaaggtggcctgaaggtcggggacggaaaggttgtactgaaccagcaggcggacaataaaggacaggtgcaggcgttcagcagtgttaatattgccagtggccggccgaccgtggtactgactgatgagcggcaggtaaatccggataccgtctcatggggatatcgtgggggcacactggatgttaatggtaacagtctgacgtttcatcagttgaaggcggcagattatggtgccgtgctggcgaataacgttgataaacgggccactatcacgctggactatgccctgcgggctgacaaagtagcactgaatggctggtcggaatcaggtaaaggaactgccggaaatttatataaatacaataacccgtacacaaatacgacggattacttcatcctgaagcagagcacctatggttatttccccacggaccagagcagcaacgccacctgggagtttgtggggcacagtcagggggatgcacagaaactggtagctgaccgtttcaatactgcagggtatctgtttcacggacaactgaaaggcaatctgaatgtggacaatcgcctgcctgaaggcgttaccggtgctctggtgatggacggagctgcggatatctccggtacattcacccaggaaaacgggcgtctgacgctgcaggggcatccggttatccatgcatacaatactcagtctgtggctgacaaactggctgccagtggagaccattcggttctgactcagcctacgtcattcagtcaggaggactgggagaaccgcagttttacctttgacaggctgtcactgaagaacactgattttggtcttggtcgcaatgccacactgaacacaaccatccaggcagataactccagcgtcacgctgggcgacgggagcgggagctccgagttaggccctgatggagatgaagaagaaactccagcgccggctcctcaaccagagcaaccagctcctgcaccaaaaccagagcaaccagctccagctccaaaaccagagcaaccagctcctgcaccaaaaccagagcaaccagctccagctccaaaaccagagcaaccagctccagctccaaaaccagagcaaccagctaagccggagaaaccagctgaagagcctactcaaccagaaaaaccagccactccaaaaacaggctggaaacaaggatccggcagcggtagtgtcttcaacggcaccgtcaacctggataatcagtcagtgctgaatatcaatgatatattcaatggcggaatacaggcgaacaacagtaccgtgaatatctcctcagacagtgccgttctggggaactcaacactgaccagtaccgccctgaatctgaacaagggagcaaatgctctggccagtcagagttttgtttctgacggtccagtgaatatttctgatgccaccctgagtctgaacagccgtcctgatgaggtatctcacacacttttacctgtatacgattatgccggttcatggaacctgaagggagacgatgcccgcctgaacgtggggccgtacagtatgttgtcaggtaatatcaatgttcaggataaagggactgtcaccctcggaggggaaggggaactgagtcctgacctgactcttcagaatcagatgttgtacagcctgtttaacgggtaccgcaatatctggagcgggagcctgaatgcaccggatgccaccgtcagcatgacagacacccagtggtcgatgaacggaaactccacggcaggaaatatgaaacttaaccggacaatagtcggttttaacgggggaacatcaccgttcacgacactgacaacagataatctggacgcggttcagtcagcatttgtcatgcgtacagaccttaacaaggcagacaaactggtgataaacaagtcggcaacaggtcatgacaacagcatctgggttaacttcctgaaaaaaccttctaacaaggacacgcttgatattccactggtcagcgcacctgaagcgacagctgataatctgttcagggcatcaacacgggttgtgggattcagtgatgtcacccccatccttagtgtcagaaaagaggacgggaaaaaagagtgggtcctcgatggttaccaggttgcacgtaacgacggccagggtaaggctgccgccacattcatgcacatcagctataacaacttcatcactgaagttggttccctgaacaaacgcatgggcgatttgagggatattaatggcgaagccggtacgtgggtgcgtctgctgaacggttccggctctgctgatggcggtttcactgaccactataccctgctgcagatgggggctgaccgtaagcacgaactgggaagtatggacctgtttaccggcgtgatggccacctacactgacacagatgcgtcagcagacctgtacagcggtaaaacaaaatcatggggtggtggtttctatgccagtggtctgttccggtccggcgcttactttgatgtgattgccaaatatattcacaatgaaaacaaatatgacctgaactttgccggagctggtaaacagaacttccgcagccattcactgtatgcaggtgcagaagtcggataccgttatcatctgacagatacgacgtttgttgaacctcaggcggaactggtctggggaagactgcagggccaaacatttaactggaacgacagtggaatggatgtctcaatgcgtcgtaacagcgttaatcctctggtaggcagaaccggcgttgtttccggtaaaaccttcagtggtaaggactggagtctgacagcccgtgccggcctgcattatgagttcgatctgacggacagtgctgacgttcatctgaaggatgcagcgggagaacatcagattaatggcagaaaagacagtcgtatgctttacggtgtggggttaaatgcccggtttggcgacaatacgcgtctggggctggaagttgaacgctctgcatttggtaaatacaacacagatgatgcgataaacgctaatattcgttattcattctga

SEQ ID NO: 18 - DNA, 4014 bp, Escherichia coli
atgaacagaatttattctcttcgctacagcgctgtggcccggggctttattgccgtatctgagtttgctaggaaatgtgttcataagtctgtcagacgtctgtgtttcccggttttattactgatcccggtactattctctgcaggaagtcttgcggggagctccatggcaaataaagcagtaaatgactttatactagctatgaattacgataaaaagaaactcttgacccatcagggagaaagtattgaaaatcgtttcatcaaagagggtaatcagctacccgatgagtttgttgttatcgaaagaaagaagcggagcttgtcgacaaatacaagtgatatttctgtaacagctaccaacgacagtcgcctctatcctggagcacttctcgtagtggatgagaccttgttagagaataatcccactcttcttgcggtcgatcgtgctccgatgacttatagtattgatttgcctggtttggcaagtagcgatagctttctccaagtggaagaccccagcaattcaagtgttcgcggagcggtaaacgatttgttggctaagtggcatcaagattatggtcaggtcaataatgtcccagctagaatgcagtatgaaaaaatcacggctcacggatccggcagcggtaatgatgccccggtcacgttcagaacatcggaaggtggtgcactggagtggagctttaacagcagtaccggagctggtgcgctgacacagggaaccaccacatatgccatgcacgggcagcagggaaatgacctgaatgctggtaagaacctgatatttcaggggcagaatggtcagattaaccttaaggattcggtttctcagggggcgggttccctgacgttccgtgataattacacagtaacaacctctaacggaagtacctggaccggtgccggtattgttgtggacaacggggtgtccgtaaactggcaggttaatggtgttaagggcgataacctgcataaaattggtgaaggtacgctgacggtacagggtacaggtattaatgaaggtggcctgaaggtcggggacggaaaggttgtactgaaccagcaggcggacaataaaggacaggtgcaggcgttcagcagtgttaatattgccagtggccggccgaccgtggtactgactgatgagcggcaggtaaatccggataccgtctcatggggatatcgtgggggcacactggatgttaatggtaacagtctgacgtttcatcagttgaaggcggcagattatggtgccgtgctggcgaataacgttgataaacgggccactatcacgctggactatgccggttctgggagctccgcttacagaaacggagatttactgctggatcatagtggtgcctatgttgcccaatattatattacttggaatgaattatcctatgatcatcaaggtaaggaagtcttgactcctaaggcttgggacagaaatgggcaggatttaacggctcactttaccactagtattcctttaaaagggaatgttcgtaatctctctgtcaaaattagagagtgtaccgggcttgcctgggaatggtggcgtacggtttatgaaaaaaccgatttgccactagtgcgtaagcggacgatttctatttggggaacaactctctatcctcaggtagaagataaggtagaaaatgacggatccggcagcggtaatactgcagggtatctgtttcacggacaactgaaaggcaatctgaatgtggacaatcgcctgcctgaaggcgttaccggtgctctggtgatggacggagctgcggatatctccggtacattcacccaggaaaacgggcgtctgacgctgcaggggcatccggttatccatgcatacaatactcagtctgtggctgacaaactggctgccagtggagaccattcggttctgactcagcctacgtcattcagtcaggaggactgggagaaccgcagttttacctttgacaggctgtcactgaagaacactgattttggtcttggtcgcaatgccacactgaacacaaccatccaggcagataactccagcgtcacgctgggcgacagccgggtatttatcgacaaaaacgatggccagggaacagcctttacccttgaagaaggcacatctgttgcaactaaagatgcagataaaagtgtcttcaacggcaccgtcaacctggataatcagtcagtgctgaatatcaatgatatattcaatggcggaatacaggcgaacaacagtaccgtgaatatctcctcagacagtgccgttctggggaactcaacactgaccagtaccgccctgaatctgaacaagggagcaaatgctctggccagtcagagttttgtttctgacggtccagtgaatatttctgatgccaccctgagtctgaacagccgtcctgatgaggtatctcacacacttttacctgtatacgattatgccggttcatggaacctgaagggagacgatgcccgcctgaacgtggggccgtacagtatgttgtcaggtaatatcaatgttcaggataaagggactgtcaccctcggaggggaaggggaactgagtcctgacctgactcttcagaatcagatgttgtacagcctgtttaacgggtaccgcaatatctggagcgggagcctgaatgcaccggatgccaccgtcagcatgacagacacccagtggtcgatgaacggaaactccacggcaggaaatatgaaacttaaccggacaatagtcggttttaacgggggaacatcaccgttcacgacactgacaacagataatctggacgcggttcagtcagcatttgtcatgcgtacagaccttaacaaggcagacaaactggtgataaacaagtcggcaacaggtcatgacaacagcatctgggttaacttcctgaaaaaaccttctaacaaggacacgcttgatattccactggtcagcgcacctgaagcgacagctgataatctgttcagggcatcaacacgggttgtgggattcagtgatgtcacccccatccttagtgtcagaaaagaggacgggaaaaaagagtgggtcctcgatggttaccaggttgcacgtaacgacggccagggtaaggctgccgccacattcatgcacatcagctataacaacttcatcactgaagttggttccctgaacaaacgcatgggcgatttgagggatattaatggcgaagccggtacgtgggtgcgtctgctgaacggttccggctctgctgatggcggtttcactgaccactataccctgctgcagatgggggctgaccgtaagcacgaactgggaagtatggacctgtttaccggcgtgatggccacctacactgacacagatgcgtcagcagacctgtacagcggtaaaacaaaatcatggggtggtggtttctatgccagtggtctgttccggtccggcgcttactttgatgtgattgccaaatatattcacaatgaaaacaaatatgacctgaactttgccggagctggtaaacagaacttccgcagccattcactgtatgcaggtgcagaagtcggataccgttatcatctgacagatacgacgtttgttgaacctcaggcggaactggtctggggaagactgcagggccaaacatttaactggaacgacagtggaatggatgtctcaatgcgtcgtaacagcgttaatcctctggtaggcagaaccggcgttgtttccggtaaaaccttcagtggtaaggactggagtctgacagcccgtgccggcctgcattatgagttcgatctgacggacagtgctgacgttcatctgaaggatgcagcgggagaacatcagattaatggcagaaaagacagtcgtatgctttacggtgtggggttaaatgcccggtttggcgacaatacgcgtctggggctggaagttgaacgctctgcatttggtaaatacaacacagatgatgcgataaacgctaatattcgttattcattctga

SEQ ID NO: 19 - DNA, 2235 bp, Streptococcus pneumoniae
atgaataagaaaaaaatgattttaacaagtctagccagcgtcgctatcttaggggctggttttgttacgtctcagcctacttttgtaagagcagaagaatctccacaagttgtcgaaaaatcttcattagagaagaaatatgaggaagcaaaagcaaaagctgatactgccaagaaagattacgaaacggctaaaaagaaagcagaagacgctcagaaaaagtatgaagatgatcagaagagaactgaggagaaagctcgaaaagaagcagaagcatctcaaaaattgaatgatgtggcgcttgttgttcaaaatgcatataaagagtaccgagaagttcaaaatcaacgtagtaaatataaatctgacgctgaatatcagaaaaaattaacagaggtcgactctaaaatagagaaggctaggaaagagcaacaggacttgcaaaataaatttaatgaagtaagagcagttgtagttcctgaaccaaatgcgttggctgagactaagaaaaaagcagaagaagctaaagcagaagaaaaagtagctaagagaaaatatgattatgcaactctaaaggtagcactagcgaagaaagaagtagaggctaaggaacttgaaattgaaaaacttcaatatgaaatttctactttggaacaagaagttgctactgctcaacatcaagtagataatttgaaaaaacttcttgctggtgcggatcctgatgatggcacagaagttatagaagctaaattaaaaaaaggagaagctgagctaaacgctaaacaagctgagttagcaaaaaaacaaacagaacttgaaaaacttcttgacagccttgatcctgaaggtaagactcaggatgaattagataaagaagcagaagaagctgagttggataaaaaagctgatgaacttcaaaataaagttgctgatttagaaaaagaaattagtaaccttgaaatattacttggaggggctgatcctgaagatgatactgctgctcttcaaaataaattagctgctaaaaaagctgagttagcaaaaaaacaaacagaacttgaaaaacttcttgacagccttgatcctgaaggtaagactcaggatgaattagataaagaagcagaagaagctgagttggataaaaaagctgatgaacttcaaaataaagttgctgatttagaaaaagaaattagtaaccttgaaatattacttggaggggctgattctgaagatgatactgctgctcttcaaaataaattagctactaaaaaagctgaattggaaaaaactcaaaaagaattagatgcagctcttaatgagttaggccctgatggagatgaagaagaaactccagcgccggctcctcaaccagagcaaccagctcctgcaccaaaaccagagcaaccagctccagctccaaaaccagagcaaccagctcctgcaccaaaaccagagcaaccagctccagctccaaaaccagagcaaccagctccagctccaaaaccagagcaaccagctaagccggagaaaccagctgaagagcctactcaaccagaaaaaccagccactccaaaaacaggctggaaacaagaaaacggtatgtggtatttctacaatactgatggttcaatggcaataggttggctccaaaacaacggttcatggtactacctaaacgctaacggcgctatggcaacaggttgggtgaaagatggagatacctggtactatcttgaagcatcaggtgctatgaaagcaagccaatggttcaaagtatcagataaatggtactatgtcaacagcaatggcgctatggcgacaggctggctccaatacaatggctcatggtactacctcaacgctaatggtgatatggcgacaggatggctccaatacaacggttcatggtattacctcaacgctaatggtgatatggcgacaggatgggctaaagtcaacggttcatggtactacctaaacgctaacggtgctatggctacaggttgggctaaagtcaacggttcatggtactacctaaacgctaacggttcaatggcaacaggttgggtgaaagatggagatacctggtactatcttgaagcatcaggtgctatgaaagcaagccaatggttcaaagtatcagataaatggtactatgtcaatggcttaggtgcccttgcagtcaacacaactgtagatggctataaagtcaatgccaatggtgaatgggtttaa

SEQ ID NO: 20 - DNA, 786 bp, Streptococcus pneumoniae
gaagaatctccacaagttgtcgaaaaatcttcattagagaagaaatatgaggaagcaaaagcaaaagctgatactgccaagaaagattacgaaacggctaaaaagaaagcagaagacgctcagaaaaagtatgaagatgatcagaagagaactgaggagaaagctcgaaaagaagcagaagcatctcaaaaattgaatgatgtggcgcttgttgttcaaaatgcatataaagagtaccgagaagttcaaaatcaacgtagtaaatataaatctgacgctgaatatcagaaaaaattaacagaggtcgactctaaaatagagaaggctaggaaagagcaacaggacttgcaaaataaatttaatgaagtaagagcagttgtagttcctgaaccaaatgcgttggctgagactaagaaaaaagcagaagaagctaaagcagaagaaaaagtagctaagagaaaatatgattatgcaactctaaaggtagcactagcgaagaaagaagtagaggctaaggaacttgaaattgaaaaacttcaatatgaaatttctactttggaacaagaagttgctactgctcaacatcaagtagataatttgaaaaaacttcttgctggtgcggatcctgatgatggcacagaagttatagaagctaaattaaaaaaaggagaagctgagctaaacgctaaacaagctgagttagcaaaaaaacaaacagaacttgaaaaacttcttgacagccttgatcctgaaggtaagactcaggatgaattagataaagaagca

SEQ ID NO: 21 - DNA, 396 bp, Streptococcus pneumoniae
gaagaatctccacaagttgtcgaaaaatcttcattagagaagaaatatgaggaagcaaaagcaaaagctgatactgccaagaaagattacgaaacggctaaaaagaaagcagaagacgctcagaaaaagtatgaagatgatcagaagagaactgaggagaaagctcgaaaagaagcagaagcatctcaaaaattgaatgatgtggcgcttgttgttcaaaatgcatataaagagtaccgagaagttcaaaatcaacgtagtaaatataaatctgacgctgaatatcagaaaaaattaacagaggtcgactctaaaatagagaaggctaggaaagagcaacaggacttgcaaaataaatttaatgaagtaagagcagttgtagttcctgaaccaaat

SEQ ID NO: 22 - DNA, 273 bp, Streptococcus pneumoniae
gtaagagcagttgtagttcctgaaccaaatgcgttggctgagactaagaaaaaagcagaagaagctaaagcagaagaaaaagtagctaagagaaaatatgattatgcaactctaaaggtagcactagcgaagaaagaagtagaggctaaggaacttgaaattgaaaaacttcaatatgaaatttctactttggaacaagaagttgctactgctcaacatcaagtagataatttgaaaaaacttcttgctggtgcggatcctgatgatggcaca

SEQ ID NO: 23 - DNA, 30 bp, ARTIFICIAL SEQUENCE
  Synthetic cloning primer
cggggagctccgagttaggccctgatggag

SEQ ID NO: 24 - DNA, 31 bp, ARTIFICIAL SEQUENCE
  Synthetic cloning primer
tgccggatccttgtttccagcctgtttttgg

SEQ ID NO: 25 - DNA, 36 bp, ARTIFICIAL SEQUENCE
  Synthetic cloning primer
cggggagctcccttgctggtgcagatcctgatgatg

SEQ ID NO: 26 - DNA, 33 bp, ARTIFICIAL SEQUENCE
  Synthetic cloning primer
tgccggatccagtttcttcttcatctccatcag

SEQ ID NO: 27 - DNA, 31 bp, ARTIFICIAL SEQUENCE
  Synthetic cloning primer
cggggagctccgtaagagcagttgtagttcc

SEQ ID NO: 28 - DNA, 37 bp, ARTIFICIAL SEQUENCE
  Synthetic cloning primer
tgccggatcctgtgccatcatcaggatctgcaccagc

SEQ ID NO: 29 - DNA, 32 bp, ARTIFICIAL SEQUENCE
  Synthetic cloning primer
cggggagctccgaagaatctccacaagttgtc

SEQ ID NO: 30 - DNA, 33 bp, ARTIFICIAL SEQUENCE
  Synthetic cloning primer
tgccggatccatttggttcaggaactacaactg

SEQ ID NO: 31 - DNA, 35 bp, ARTIFICIAL SEQUENCE
  Synthetic cloning primer
cggggagctccatggcaaataaagcagtaaatgac

SEQ ID NO: 32 - DNA, 32 bp, ARTIFICIAL SEQUENCE
  Synthetic cloning primer
tgccggatccgtgagccgtgattttttcatac

SEQ ID NO: 33 - DNA, 35 bp, ARTIFICIAL SEQUENCE
  Synthetic cloning primer
cggggagctccgcttacagaaacggagatttactg

SEQ ID NO: 34 - DNA, 34 bp, ARTIFICIAL SEQUENCE
  Synthetic cloning primer
tgccggatccgtcattttctaccttatcttctac

SEQ ID NO: 35 - DNA, 66 bp, ARTIFICIAL SEQUENCE
  Synthetic cloning primer
aaaccatgggccatcatcatcatcatcatcatcacagcagcggcgaagaatctccacaagttgtcg

SEQ ID NO: 36 - DNA, 29 bp, ARTIFICIAL SEQUENCE
  Synthetic cloning primer
tttcatatgttaaacccattcaccattgg

(19) United States
(12) Patent Application Publication
(10) Pub. No.: US 2017/0258885 A1
(43) Pub. Date: Sep. 14, 2017

(54) FUSION PROTEIN COMPRISING STREPTOCOCCAL ANTIGEN

(71) Applicants: Abera Bioscience AB, Stockholm (SE); STICHTING KATHOLIEKE UNIVERSITEIT, Nijmegen (NL)

(72) Inventors: Joen LUIRINK, Amsterdam (NL); Wouter Simon Petrus JONG, Amsterdam (NL); Marinus Isaäk DE JONGE, Nijmegen (NL); Kirsten KUIPERS, Rijssen (NL)

(21) Appl. No.: 15/457,720

(22) Filed: Mar. 13, 2017

Related U.S. Application Data
(60) Provisional application No. 62/308,115, filed on Mar. 14, 2016.

(51) Int. Cl. A61K 39/09; C12N 9/52; C07K 14/315

(52) U.S. Cl. CPC A61K 39/092; C07K 14/3156; C12N 9/52; C12Y 304/21; C07K 2319/40; C07K 2319/033; A61K 2039/543

(57) ABSTRACT

The disclosure provides a fusion protein comprising at least one antigenic fragment of a protein from a bacterium from genus Streptococcus, as well as means for its expression. Outer membrane vesicles and vaccines comprising the fusion protein are also disclosed, as well as a method of vaccination using such vaccines.

### FIELD OF THE DISCLOSURE

The present disclosure concerns novel fusion proteins, useful for example in vaccines for protection against bacteria from genus Streptococcus.

### REFERENCE TO SEQUENCE LISTING

A Sequence Listing submitted as an ASCII text file via EFS-Web is hereby incorporated by reference in accordance with 35 U.S.C. §1.52(e). The name of the ASCII text file for the Sequence Listing is 25464280_1.TXT, the date of creation of the ASCII text file is Mar. 13, 2017, and the size of the ASCII text file is 131 KB.

### BACKGROUND

Outer membrane vesicles (OMVs), ubiquitously released from the outer membrane (OM) of Gram-negative bacteria, are promising as vaccines because they combine antigen and adjuvant in a single formulation. The intrinsic adjuvant activity provided by the presence of various pathogen recognition receptor ligands, such as lipopolysaccharide and immunogenic surface proteins, forms an attractive combination with the non-living, particulate nature of the OMVs. OMVs have protected animals against various pathogens, and a licensed OMV vaccine against Neisseria meningitidis has been proven safe and protective in humans.

Accumulating evidence indicates that both the magnitude and the breadth of the immune response can be improved by secreting or displaying antigens at the surface of bacterial and viral vaccine vectors. Recently, the Escherichia coli autotransporter (AT) hemoglobin protease (Hbp) was engineered into a platform for efficient display of heterologous polypeptides at the surface of live bacteria (Jong et al (2012) Microb Cell Fact 11:85; Oloo et al (2011) J Biol Chem 286(14):12133-40), bacterial ghosts (Hjelm et al (2014) Appl Environ Microbiol 81(2):726-35) and OMVs (Daleke-Schermerhorn et al (2014) Appl Environ Microbiol 80(18):5854-65). The AT pathway is the most widespread system for transport of proteins across the Gram-negative cell envelope, employing a relatively simple two-step mechanism. The AT is first transported across the inner membrane by the Sec machinery, after which its C-terminal domain inserts into the OM and forms a β-barrel that together with a central linker domain mediates transport of the functional N-terminal passenger domain to the cell surface or medium (van Ulsen et al (2014) Biochim Biophys Acta 1843(8):1592-611). Based on the available crystal structure of the Hbp passenger (Otto et al (2005) J Biol Chem 280(17):17339-45), a side-domain replacement strategy was developed that allows fusion of multiple heterologous sequences to a single, stable Hbp scaffold (Jong et al (2012) supra; Jong et al (2014) Microb Cell Fact 13:162; WO2012/041899). Using various mycobacterial, chlamydial and influenza antigens, the Hbp platform was demonstrated to be a versatile tool for the simultaneous display of multiple sizeable antigens at the surface of live bacteria and OMVs (Jong et al (2014) supra; WO2012/041899; Daleke-Schermerhorn et al (2014) supra).

Current vaccines against streptococci, such as pneumococcal conjugate vaccines (PCVs), consisting of capsular polysaccharides, have strongly reduced the incidence of severe disease caused by vaccine-specific S. pneumoniae serotypes (Feldman and Anderson (2014) J Infect 69(4):309-25). However, the efficacy of PCVs is reduced by serotype replacement (Hicks et al (2007) J Infect Dis 196(9):1346-54; Lexau et al (2005) JAMA 294(16):2043-51; Miller et al (2011) Lancet Infect Dis 11(10):760-8) and capsular switching events (Brueggemann et al (2007) PLoS Pathog 3(11):e168), and their accessibility in developing countries is restricted by complex, costly manufacturing and the requirement for needle-based administration. Thus, there is a need in the art of alternative streptococcal vaccines that employ novel approaches.

### SUMMARY OF THE INVENTION

One object of the disclosure is to provide a streptococcal vaccine based on non-capsular protein antigens.

Another object of the disclosure is to provide a streptococcal vaccine which is suitable for various modes of administration, in particular mucosal administration, for example intranasal administration.

Another object of the disclosure is to provide protective immunity in vivo by a vaccine formulation comprising autotransporter-based fusion proteins.

Another object of the disclosure is the evaluation and comparison of different candidate protein antigens for a streptococcal vaccine.

Another object of the disclosure is the provision of an OMV-based streptococcal vaccine which elicits an immune response with a beneficial cytokine profile.

Another object of the disclosure is to provide a fusion protein which is suitable for use in a streptococcal vaccine, for example an OMV-based streptococcal vaccine.

These, and other objects which are evident to the skilled person from the present disclosure, are met by the different aspects of the invention as claimed in the appended claims and as generally disclosed herein.

Thus, in a first aspect, the disclosure provides a fusion protein, comprising

i. a passenger domain comprising a beta stem domain from an autotransporter protein, wherein the beta stem forming sequence of the passenger domain is essentially intact;

ii. a translocator domain from an autotransporter protein;

iii. a signal peptide that targets the fusion protein to the inner membrane of a Gram negative bacterium; and

iv. at least one antigenic fragment;

wherein the passenger domain of the autotransporter in its native form comprises at least one side domain, and wherein said antigenic fragment replaces or partly replaces said side domain; and

wherein said at least one antigenic fragment is a fragment of a protein from a bacterium of the genus Streptococcus.

The replacement of at least one side domain of a passenger domain in a fusion protein has been demonstrated previously, for example in the PCT application published as WO2012/041899, which is hereby incorporated by reference in its entirety, or in the articles by Jong et al (2012), supra, Jong et al (2014) supra, and by Daleke-Schermerhorn et al (2014), supra. The teachings therein concerning the structural features i.-iii. of the fusion protein apply equally to the present disclosure.

The fusion protein of the present disclosure comprises an antigenic fragment, which is a fragment of a protein from a bacterium of the genus Streptococcus. In the work on the invention, the inventors have surprisingly shown that the display of streptococcal antigens as part of the disclosed fusion protein on the surface of outer membrane vesicles has the potential to elicit a protective immune response based on IL-17A secretion. In one embodiment, the at least one antigenic fragment is a fragment of a protein from an alpha-hemolytic Streptococcus bacterium. In another embodiment, the at least one antigenic fragment is a fragment of a protein from a beta-hemolytic Streptococcus bacterium.

In a more specific embodiment, the fusion protein comprises at least one antigenic fragment, which is a fragment of a protein from Streptococcus pneumoniae. In one embodiment, this at least one antigenic fragment is a fragment of pneumococcal surface protein A (PspA) from Streptococcus pneumoniae.

In an even more specific embodiment, the at least one antigenic fragment is a fragment of the α-helical coiled-coil domain of pneumococcal surface protein A (PspA) from Streptococcus pneumoniae. Such a PspA protein may for example be PspA from strain TIGR4 of S. pneumoniae (SEQ ID NO:8). Surprisingly, the use of such an antigenic fragment in the fusion protein according to this aspect of the disclosure was demonstrated by the inventors (see Example below) to be superior with respect to the induction of protection against pneumococcal colonization.

In one embodiment, the at least one antigenic fragment is a fragment of an α-helical coiled-coil domain in PspA which extends from the end of the N-terminal signal sequence to the beginning of the lactoferrin-binding domain (LFBD) (see FIG. 1). As demonstrated in the Example below, the inventors have shown that antigenic fragments from this N-terminal domain, as opposed to the LFBD fragment, are particularly suited as antigenic fragments in a fusion protein as disclosed herein.

In a more specific embodiment, said α-helical coiled-coil domain from PspA comprises an amino acid sequence selected from the group consisting of SEQ ID NO:9 and antigenic fragments thereof, and homologous sequences having at least 30% identity thereto.

In another embodiment, the at least one antigenic fragment is 20-250 amino acids in length, such as 50-250 amino acids, for example 75-225 amino acids. Antigenic fragments in this size range are found to be particularly suitable for expression as replacement side domains in the fusion proteins of this aspect of the disclosure.

As further disclosed in WO2012/041899, an autotransporter passenger domain may, in its native form, comprise more than one side domain that can be replaced by a protein of interest. In some embodiments of the present disclosure, this fact is exploited by introducing two or more, overlapping or non-overlapping, antigenic fragments at separate side domain positions on the passenger domain. In other words, one embodiment of this aspect of the disclosure is a fusion protein as described above, comprising at least a first antigenic fragment α1 and a second antigenic fragment α2 which are different, overlapping or non-overlapping, fragments of said α-helical coiled-coil domain of pneumococcal surface protein A from Streptococcus pneumoniae, wherein the passenger domain of the autotransporter in its native form comprises at least two side domains, and wherein each of said antigenic fragments α1 and α2 replaces or partly replaces a separate side domain.

In one embodiment, the fragments α1 and α2 are different fragments from that part of the α-helical coiled-coil domain in PspA which extends from the end of the N-terminal signal sequence to the beginning of the lactoferrin-binding domain (LFBD).

With regard to the antigenic fragment α1 in these embodiments of the disclosure, it may for example be a fragment which is N-terminal to the antigenic fragment α2 in the PspA protein.

In one embodiment, the amino acid sequence of α1 consists of 100-150 amino acid residues, for example from 120-140 amino acid residues, for example 125-135 amino acid residues.

In another embodiment, the amino acid sequence of α1 comprises a sequence selected from the group consisting of SEQ ID NO:10 and sequences having at least 30% identity thereto.

In a more specific embodiment, said amino acid sequence comprises SEQ ID NO:10.

In another more specific embodiment, said amino acid sequence of α1 consists of a sequence selected from the group consisting of SEQ ID NO:10 and sequences having at least 30% identity thereto. For example, the α1 sequence may consist of SEQ ID NO:10.

With regard to the antigenic fragment α2 in these embodiments of the disclosure, it may for example be a fragment which is C-terminal to the antigenic fragment α1 in the PspA protein.

In one embodiment, the amino acid sequence of α2 consists of 60-110 amino acid residues, for example 70-100 amino acid residues, for example 75-95 amino acid residues.

In another embodiment, the amino acid sequence of α2 comprises a sequence selected from the group consisting of SEQ ID NO:11 and sequences having at least 30% identity thereto.

In a more specific embodiment, said amino acid sequence comprises SEQ ID NO:11.

In another more specific embodiment, said amino acid sequence of α2 consists of a sequence selected from the group consisting of SEQ ID NO:11 and sequences having at least 30% identity thereto. For example, the α2 sequence may consist of SEQ ID NO:11.

Alignment of sequences homologous to SEQ ID NO:9, 10 or 11 from various strains of Streptococcus pneumoniae demonstrates that this section of the PspA protein exhibits diversity, such that an identity of at least 30% offers a reasonable generalization of the homologous sequences found in this region. In some embodiments, the identity may be higher, for example at least 35%, at least 40%, at least 45%, at least 50%, at least 55%, at least 60%, at least 65%, at least 70%, at least 75%, at least 80%, at least 85%, at least 90% or at least 95% identity to SEQ ID NO:9, 10 or 11.

With regard to structural elements of the fusion protein that are derived from an autotransporter protein, the following discussion applies.

As disclosed in e.g. WO2012/041899, an autotransporter protein can be used as a basis for the fusion protein of this disclosure if the beta stem forming sequence of the passenger domain of the autotransporter is essentially intact. Whereas the actual beta stem-forming sequence is essential for optimal secretion, the side domains of the passenger domain of autotransporters are suitable sites for the insertion of at least one antigenic fragment of PspA. The antigenic fragment can be inserted so as to completely replace the native side domain, so as to replace only a part of the side domain, or so as to be fused to the side domain.

As indicated above, many autotransporter proteins can be used for insertion of more than one, such as at least two, antigenic fragments, always provided that the beta stem forming sequence of the passenger domain of the autotransporter is kept essentially intact. By fusing, i.e. inserting, replacing or partly replacing, antigenic fragments to one or more side domains of the passenger, while keeping the beta stem structure intact, an efficient and relatively easy-to-use system for simultaneous display of two or more antigenic fragments is possible.

Side domains that can be replaced are relatively large, such as 20, 30, 40, 60, 80 or more amino acid residues.

Thus, a native passenger domain of an autotransporter protein can be considered as comprising several sections of beta stem forming sequence, linked together by non-beta stem forming sequences. The non-beta stem forming sequences are suitable sites for insertion of one or more antigenic fragments. Thus, an antigenic fragment can be placed between two parts of beta stem forming sequence. The antigenic fragment can also be fused to the N-terminus of the passenger domain.

Suitable methods for detecting beta stem forming sequence and side domains of passenger domains of autotransporters include biophysical methods such as as x-ray crystallography and bioinformatics software such as structure prediction tools. X-ray crystallography is a standard procedure that is highly efficient and automated, and familiar to a person skilled in the art. Examples of high resolution structures of passenger domains and suitable methods for determination of structures of the passenger domain of autotransporters are e.g. found in Otto et al (2005), supra; Emsley et al (1996) Nature 381:90-92; and Johnson et al (2009) J Mol Biol 389(3):559-74.

An example of a bioinformatics method that is suitable for determining beta stem structure is the M4T homology modeling method (Rykunov et al 2009 J Struct Funct Genomics 10: 95-99).

Where a three-dimensional model of the protein is used for the identification of beta stem domains and side domains, it is suitable that the model obtained has a resolution of better than 4 angstrom. Side domains will then be visible as domains that protrude from the beta stem. By observation of the structure of the passenger domains of autotransporters, it can be seen that parts of the sequence are not part of the beta stem but form domains that protrude from the beta stem.

Methods such as those described above can be used for determining which domains or amino acids of the native passenger domains are suitable for insertion of an antigenic fragment and which should be kept essentially intact.

In the fusion protein according to the disclosure, the beta stem forming sequence from the autotransporter passenger domain is essentially intact. Thus, as little as possible of the beta stem forming sequence should be removed. Predicted domain borders are of help to determine where an antigenic fragment can be inserted. If too much of the beta stem forming sequence is removed, secretion will be negatively affected. That the beta stem forming sequence is essentially intact means that the efficiency of the secretory function of the protein is maintained at an optimal level, as compared to when the beta stem is disrupted or completely removed. It also means that the stability of the passenger after secretion is maintained. A person skilled in the art can use experimental methods to determine if a particular construct allows for efficient secretion.

Examples of methods suitable for determining the efficiency of secretion in vitro include: analysis of the fraction of antigenic fragment present in the medium, labeling of surface proteins with biotin or other labels, cell fractionation, exposure of surface proteins to proteases (such as proteinase K) and studies using antibodies against the antigenic fragments (such as dot blot studies, immunofluorescence microscopy and immuno-electron microscopy).

Examples of methods suitable for determining the stability of the passenger after secretion include SDS-PAGE, western blotting, as well as the methods described in the preceding paragraph.

Thus, by using structural information, a person skilled in the art can predict where in the passenger domain the insertion of the antigenic fragment can be made in order to maintain optimal secretion. Actual secretion can be easily determined with in vitro experiments.

In the disclosed fusion protein, the at least one antigenic fragment is fused to the passenger domain. This means that the antigenic fragment is fused to the peptide that forms the passenger domain such that they form one continuous polypeptide. Because design of a fusion protein is done at the DNA level, care must be taken so that the reading frame of the antigenic fragment is the same as the reading frame of the passenger domain.

In one embodiment, a fusion protein as disclosed herein comprises a passenger domain from one type of autotransporter and a translocator domain from the same type of autotransporter.

In another embodiment, a fusion protein as disclosed herein comprises a passenger domain from one type of autotransporter and a translocator domain from another type of autotransporter.

An autotransporter protein used as a basis for the disclosed fusion protein can be an autotransporter with a serine protease domain, such as a serine protease.

The autotransporter can be a SPATE protein (Serine protease autotransporters of Enterobacteriaceae). Thus, the translocator domain and the passenger domain can be from a SPATE protein.

The SPATE group of proteins has several advantages for use as a basis for the fusion protein of the disclosure. First of all, some of their structures are known, which facilitates the identification of beta stem and side domains in the passenger domain. Structural knowledge can also be used for prediction of side domains and beta stem structures in related SPATEs, for which the crystal structure is not known. Another advantage lies in the cleavage structure, which can be used for efficient soluble secretion and is conserved within the SPATE family.

Even though SPATE autotransporters are considered to be suitable in connection with the present disclosure, other autotransporters, for which the structure is known or can be predicted or ascertained such that their beta stem and side domain structure is determined, may also be used as basis for the disclosed fusion protein. Such an autotransporter should have a beta stem, at least one side domain and optionally a cleavage system that is efficient for soluble secretion. One example is the autotransporter HapS from H. influenzae, which is not a member of the SPATE family. The structure of the passenger of HapS has been published (Meng et al (2011) EMBO J 30(18):3864-74). The structure is very close to that of Hbp, having a beta-stem with four side domains.

In one embodiment, the SPATE protein is selected from the group consisting of hemoglobin-binding protease (Hbp; SwissProt 088093), extracellular serine protease (EspC) and temperature-sensitive hemagglutinin (Tsh; SwissProt Q47692) from Escherichia coli. The sequence of Tsh is highly homologous to that of Hbp.

Other contemplated SPATE proteins include IgA protease of Neisseria gonorrhoeae and Haemophilus influenzae, Pet from E. coli, EspP from E. coli, Pic from E. coli, PicU from E. coli, Sat from E. coli, Vat from E. coli, EspI from E. coli, EaaA from E. coli, EaaC from E. coli, EatA from E. coli, EpeA from E. coli, PssA from E. coli, AidA_B7A from E. coli, Boa from Salmonella bongori, SepA from Shigella flexneri, SigA from Shigella flexneri, Pic from Shigella flexneri.

In one embodiment of the fusion protein, the autotransporter is a SPATE protein comprising an amino acid sequence selected from SEQ ID NO:1, which is Hbp, SEQ ID NO:2, which is Hbp where the cleavage site between the translocator domain and the passenger domain has been disrupted (Hbp(Δβ-cleav)), and sequences that are homologous to those sequences. Based on alignments of sequences of known autotransporters with the required structure, the skilled person knows from the scientific literature that homologous autotransporters may have a sequence identity which is from 35% and up. Thus, such an homologous sequence may for example be at least 35%, at least 40%, at least 45%, at least 50%, at least 55%, at least 60%, at least 65%, at least 70%, at least 75%, at least 80%, at least 85%, at least 90% or at least 95% identical to SEQ ID NO:1 or 2.

In a specific embodiment of the fusion protein according to this aspect of the disclosure, in which the autotransporter is Hbp, the passenger domain in its native form comprises five side domains, defined by amino acids 54-308, 533-608, 657-697, 735-766 and 898-922 of SEQ ID NO:1 or SEQ ID NO:2. In an embodiment of such a fusion protein, one of said α1 and α2, when present, is inserted into, replaces or partly replaces the side domain defined by amino acids 54-308, and the other one of said α1 and α2, when present, is inserted into, replaces or partly replaces the side domain defined by amino acids 533-608.

In one embodiment, the passenger domain (i) comprises the amino acid sequence spanning positions 53-1100 of SEQ ID NO:1.

In one embodiment, the translocator domain (ii) comprises the amino acid sequence spanning positions 1101-1377 of SEQ ID NO:1.

The fusion protein further comprises an N-terminal signal peptide that directs the protein for secretion. In a Gram negative bacterial host cell, the signal peptide is suitably such that it directs translocation of the protein across the inner membrane. The signal peptide can be derived from an autotransporter protein, suitably the same autotransporter from which the passenger domain is derived. In one embodiment, the signal peptide comprises approximately amino acids 1-52 of SEQ ID NO:1, or a similar sequence.

In one embodiment, the fusion protein comprises an autochaperone domain, suitably from the passenger domain of the autotransporter protein used to fuse the POI. One example of an autochaperone domain comprises approximately amino acids 1002-1100 of SEQ ID NO:1.

A second aspect of this disclosure provides a polynucleotide encoding a fusion protein according to the first aspect. In other words, the fusion protein is encoded by a nucleic acid, which for example enables it to be expressed in a host cell. Said polynucleotide or nucleic acid can be constructed with the use of standard molecular biology techniques involving restriction enzymes, DNA ligases, PCR, oligonucleotide synthesis, DNA purification and other methods well-known to a person skilled in the art. Preferably, the starting point is a reading frame of an autotransporter protein into which a DNA fragment encoding the at least one antigenic fragment is inserted so that the reading frames match. Alternatively, the reading frame for the fusion protein can be designed in silico and synthesized using polynucleotide synthesis.

The reading frame encoding the fusion protein is preferably inserted in an expression vector for prokaryote expression carrying a promoter and other components well known to a person skilled in the art. A third aspect of the disclosure provides an expression vector comprising a polynucleotide according to the second aspect.

In a fourth aspect of the disclosure, there is provided a Gram-negative bacterial host cell, which comprises an expression vector according to the third aspect. The cell is preferably a host cell that can be cultured and manipulated by methods well known to a person skilled in the art and which is able to express heterologous proteins. In one embodiment, the cell belongs to the family Enterobacteriaceae, Preferably, the host cell is selected from the group consisting of E. coli, Salmonella spp., Vibrio spp., Shigella spp., Pseudomonas spp., Burkholderia spp. and Bordetella spp. A wide variety of expression systems are available and known to a person skilled in the art. The expression may be of a stable or transient nature. The expression system may be inducible or non-inducible.

In one embodiment, the fusion protein is designed in such a way that it is displayed at the cell surface after having been expressed and secreted. For instance, the fusion protein may comprise no cleavage site, or may comprise a disrupted cleavage site. Alternatively, the fusion protein and nucleic acid may comprise a cleavage site and the resulting fusion protein be cleaved, but remains non-covalently attached to, and thus displayed at, the cell surface.

In one embodiment, said host cell belongs to the species Salmonella enterica, for example the subspecies Salmonella enterica subsp enterica, in particular the serovar Typhimurium, for example the strain SL3261. In a specific embodiment, said host cell is a ΔtoIRA derivative of S. Typhimurium strain SL3261. As will be explained further below, expression in this host cell is beneficial in that it has the ability to shed large amounts of outer membrane vesicles displaying the fusion protein expression products on their surface, provided that the fusion protein is designed for being covalently or non-covalently attached to the cell surface. Other host cells with similar properties of OMV shedding are known to the skilled person and contemplated as useful in connection with fusion protein expression according to this disclosure.

Thus, yet another aspect of the disclosure provides a method of expressing a fusion protein as described herein, comprising the steps of

i. providing a host cell according to the fourth aspect, and

ii. culturing said host cell under conditions suitable for expression of said fusion protein.

With a suitable choice of host cell, the ΔtoIRA derivative of S. Typhimurium strain SL3261 discussed above being a non-limiting example, another aspect of the disclosure further provides a method of producing outer membrane vesicles displaying a fusion protein as described herein on their surface. This method comprises expression of the fusion protein, using the method described in the preceding paragraph, followed by actively inducing or passively allowing the host cell to shed vesicles from the outer membrane of the host cell to obtain OMVs displaying the fusion protein on their surface.

Thus, under certain conditions, Gram negative bacteria may shed vesicles from their outer membrane. Such outer membrane vesicles (OMVs) have been shown to be useful as vaccine platforms. When carrying antigens, as derived from their mother cells, these vesicles are capable of enhancing the immunogenicity of such antigen. OMVs may easily be derived from Gram negative bacteria displaying the fusion protein of the invention on their surface. Methods for OMV production and isolation are known in the art (Chen et al (2010) PNAS 107:3099-3104; Bernadac et al (1998) J Bacteriol 180: 4872-4878; Kesty and Kuehn (2004) J Biol Chem 279: 2069-2076; Kolling and Matthews (1999) App Env Microbiol 65:1843-1848; Kitagawa et al (2010) J Bacteriol 192:5645-5656).

In yet another of its aspects, the disclosure provides an outer membrane vesicle displaying at least one fusion protein as described herein on its surface.

In another aspect, the disclosure provides a vaccine that comprises such an OMV. In a related aspect, the disclosure provides a method of using such a vaccine. In other words, a method is provided for vaccination, or of inducing protective immunity, against Streptococcus, such as for example Streptococcus pneumoniae, comprising the step of administering a vaccine as disclosed herein to a subject in need thereof. In one embodiment, the protective immunity manifests itself in a protection against streptococcal colonization, such as against the colonization of Streptococcus pneumoniae, for example in the nasopharyngeal area.

The inventors have surprisingly shown that the use of the disclosed at least one antigen fragment in a fusion protein displayed on OMVs and used as a vaccine, leads to an immune response which is characterized by a beneficial cytokine profile, in the form of a high expression of IL-17A. The importance of IL-17A expression in mucosal immunity to Streptococcus has been previously shown. Thus, in one embodiment of the vaccination method of the present disclosure, the protective immunity is characterized by a high expression of IL-17A. In a more specific embodiment, said expression is localized in the nasopharyngeal tissue. Because of the observed suitability of the disclosed vaccine in protecting against mucosal colonization by Streptococcus, one embodiment of the vaccination method comprises mucosal administration of the vaccine, for example intranasal administration.

In one embodiment of the disclosed method, protective immunity is induced against a disease caused by streptococcal infection, for example selected from the group consisting of pneumonia, endocarditis, meningitis, otitis media, bacteremia, sepsis, pharyngitis, respiratory infections, dental caries and acute exacerbations of chronic bronchitis, sinusitis, arthritis and conjunctivitis.

In a more specific embodiment of the disclosed method, protective immunity is induced against a pneumococcal disease selected from the group consisting of pneumonia, meningitis, otitis media, bacteremia, sepsis and acute exacerbations of chronic bronchitis, sinusitis, arthritis and conjunctivitis.

The following definitions are supplied in order to facilitate the understanding of the disclosure:

An “autotransporter” is a protein that belongs to the pfam autotransporter family (‘Autotransporter’ PF03797) and that is also known or predicted to form a beta stem motif. The BETAWRAPPRO method for sequence analysis can be used to predict if the passenger domain of an autotransporter will form a beta stem motif (Junker et al (2006) Proc Natl Acad Sci USA 103(13):4918-23).

“Beta stem forming sequence” refers to the sequence of a passenger domain of an autotransporter that forms a beta stem structure. The beta stem forming sequence of a passenger can for example be identified using crystal structure determination. As described above the beta stem forming sequence may alternatively be identified using the M4T homology modeling method (Rykunov et al (2009), supra) or similar prediction methods.

A “side domain” is a domain that is part of the passenger domain but is not part of the beta stem. Typically, a side domain is located in the passenger domain between two stretches of beta stem forming sequence. A side domain starts at the first amino acid after the preceding beta strand and ends one amino acid before the starting amino acid of the beta strand following the side domain. A side domain can also be located at the N-terminus of the passenger domain. Autotransporters may have several side domains.

“Similar protein”, “similar sequence” or a “like protein” refers to a protein that has a high degree of homology to another protein when the two amino acid sequences are compared. With regard to the homologous proteins and fragments disclosed herein, it is at least 35%, at least 40%, at least 45%, at least 50%, at least 55%, at least 60%, at least 65%, at least 70%, at least 75%, at least 80%, at least 85%, at least 90% or at least 95% identical to the comparative sequence when the two sequences are optimally aligned. Sequence homology can be readily measured using public available software such as BLAST.

“Host cell” refers to a prokaryotic cell into which one or more vectors or isolated and purified nucleic acid sequences of the invention have been introduced. It is understood that the term refers not only to the particular subject cell but also to the progeny or potential progeny of such a cell. Because certain modifications may occur in succeeding generations due to either mutations or environmental influences, such progeny may not, in fact, be identical to the parent cell, but are still included within the scope of the term as used herein.

“Displayed”: A secreted fusion protein is displayed on the surface of the secreting host cell when it remains associated with the outer membrane of the host cell such that it at least partly protrudes outside the cell. The secreted protein may be attached to the cell membrane or a component that resides therein (such as the translocator domain from an autotransporter) in a covalent or non-covalent manner.

The following Table 1 shows different constructs that are disclosed or discussed herein. The amino acid sequences and nucleic acid sequences are listed in the appended sequence listing.

TABLE 1
Constructs

| | Amino acid sequence | Nucleic acid sequence |
|---|---|---|
| Designation | SEQ ID NO | SEQ ID NO |
| Hbp (wildtype) | 1 | 12 |
| Hbp(Δβ-cleav) | 2 | 13 |
| HbpD | 3 | 14 |
| HbpD-PspA[α1-α2] | 4 | 15 |
| HbpD-PspA[PRR] | 5 | 16 |
| HbpD-PspA[LFBD-PRR] | 6 | 17 |
| HbpD-Ply[F1-F3] | 7 | 18 |
| TIGR4 PspA | 8 | 19 |
| TIGR4 PspA aa 32-293 | 9 | 20 |
| TIGR4 PspA α1 | 10 | 21 |
| TIGR4 PspA α2 | 11 | 22 |

### BRIEF DESCRIPTION OF THE DRAWINGS

FIG. 1 is a schematic illustration of the vaccine design described in the Example. A: schematic representations of PspA and pneumolysin. The slightly overlapping fragments that were selected for fusion to HbpD cover the α-helical coiled coil domain (α1 and α2), the lactoferrin-binding domain (LFBD) and the Pro-rich region (PRR) but not the cell wall-anchoring choline-binding domain (CBD) of PspA, and the entire Ply sequence. Numbers above the diagrams and boundaries next to the fragments correspond to aa positions calculated from the N termini of full-length PspA and Ply. B: Wild-type Hbp (included for reference) comprises an N-terminal cleavable signal sequence (ss), a secreted passenger domain, and a linker (grey) and a C-terminal domain that inserts into the OM as a β-barrel, which together facilitate translocation of the passenger. Side domains d1-d5, which are dispensable for secretion and can be replaced by heterologous polypeptides, are indicated, while the remainder of the passenger domain is black. Point mutations D1100G and D1101G (denoted X) prevent autocatalytic cleavage after translocation across the OM, creating a surface-exposed (display; D) version of Hbp (Jong et al (2007) Mol Microbiol 63(5):1524-36). Numbers above the diagrams correspond to the aa positions of the wt Hbp precursor, calculated from the N terminus. Insertion of the pneumococcal PspA fragments, defined in A, are highlighted. Each insert is flanked by short flexible Gly/Ser linkers (FL).

FIG. 2 are gel photographs showing the result of vaccine production. A: SDS-PAGE/Coomassie analysis of the vaccine stocks used. Equivalent volumes of vaccine stock containing equivalent amounts of OMVs, based on OD units of the original cultures, were isolated from S. Typhimurium SL3261 ΔtoIRA expressing HbpD or the indicated HbpD-antigen chimera and loaded. All constructs were expressed in the presence of 100 μM IPTG (unlabeled or H; high), except for HbpD-PspA[α1-α2] L (low), which was expressed in the presence of 1 μM IPTG, as determined by densitometric analysis. Below the panel, relative amounts of Hbp chimeras and OmpA in OMV samples are displayed as determined by densitometric analysis. Highest measured densities were put at 100%. B-C: Equal amounts of intact (−tx) and Triton X-100-permeabilized (+tx) OMVs described in A were incubated with Proteinase K (+pk) or mock treated (−pk), separated by SDS-PAGE and detected with B: immunoblotting using indicated antibodies, and C: Coomassie staining. An amount of 0.5 OD units of OMV material was loaded in each lane. The Hbp (chimeras) are marked by asterisks, and the OMV marker proteins OmpF/C and OmpA, and Proteinase K are indicated with arrowheads.

FIG. 3 demonstrates that OMV/Hbp platform-induced protection is influenced by choice of antigen fragment, antigen amount, and immunization number. Bacterial recovery of S. pneumoniae from nasal tissue three days post intranasal challenge of C57BL/6 mice that received A: three intranasal immunizations with OMVs displaying HbpD, HbpD-PspA[α1-α2], HbpD-PspA[LFBD-PRR] or HbpD-Ply[F1-F3], or B: three, two or one intranasal immunization(s) (3×; 2×; 1×) with OMVs expressing HbpD or high (H) or 7-fold lower (L) levels of HbpD-PspA[α1-α2]. Symbols indicate individual mice (n=5-15 per group), bars represent group mean and the dotted line indicates the lower limit of detection. *, p<0.05; **, p<0.01; ***, p<0.001.

FIG. 4 shows that protective immunity correlates with intranasal IL-17A levels. A-B: Nasopharyngeal A: IFNγ and B: IL-17A three days post-infection in mice immunized three times with OMVs expressing HbpD or HbpD-PspA[α1-α2]. C: Nasopharyngeal IL-17A three days post-infection in mice immunized three, two or one times (3×; 2×; 1×) with OMVs expressing HbpD or high (H) or 7-fold lower (L) levels of HbpD-PspA[α1-α2]. A-C, *, p<0.05; **, p<0.01; ***, p<0.001. D, Pooled data from study 1 and 2 for nasopharyngeal IL-17A and number of CFU three days post-infection of mice. Symbols represent individual mice (n=5-15 per group) immunized three times with OMV-HbpD (filled symbols) or with OMV-HbpD-PspA[α1-α2] (open symbols), and bars represent group mean. Spearman's correlation coefficient (ρ) and p-value are indicated.

FIG. 5 shows that intranasal antigen-specific IgG is influenced by antigen amount. Nasal antigen-specific IgG responses directed against the whole protein, i.e. PspA and pneumolysin, in mice immunized A: three times with OMVs displaying the indicated HbpD (chimera), or B: three, two or one times (3×; 2×; 1×) with OMVs displaying HbpD, or HbpD-PspA[α1-α2] with varying antigen amount (H; high or L; 7-fold lower). Symbols represent individual mice, and bars represent mean of groups of 5-15 mice. *, p<0.05; **, p<0.01; ***, p<0.001.

FIG. 6 illustrates bacterial recovery upon high expression of PRR. Bacterial recovery of S. pneumoniae from nasal tissue three days post intranasal challenge of C57BL/6 mice that received three (3×) intranasal immunizations of OMVs displaying HbpD or high (H) levels of antigen fragment PRR. Each symbol represents an individual mouse, and bars represent mean per group. NB: The control samples (HbpD) are the same as shown in FIG. 2B.

FIG. 7 shows systemic antigen-specific IgG levels. Systemic antigen-specific IgG responses directed against PspA or pneumolysin, respectively, in mice immunized with OMVs displaying A: indicated Hbp (derivatives) or B: HbpD or HbpD-PspA[α1-α2] at high (H) or 5-fold lower (L) expression levels, and with varying immunization number (3×; 2×; 1×). Bars represent mean per group, and symbols represent samples from individual mice. *, p<0.05; **, p<0.01; *** p<0.001.

FIG. 8 shows nasal antigen-specific IgA levels. Nasal antigen-specific IgA responses directed against PspA or pneumolysin, respectively, in mice immunized with OMVs displaying, A: indicated Hbp (derivatives) or B: HbpD or HbpD-PspA[α1α2] at high (H) or 5-fold lower (L) expression levels, and with varying immunization number (3×; 2×; 1×). Bars represent mean per group, and symbols represent samples from individual mice. *, p<0.05; **<0.01; *** p<0.001.

### EXAMPLE

### Summary

This Example demonstrates the feasibility of using the autotransporter Hbp platform, designed to efficiently and simultaneously display multiple antigens at the surface of bacterial OMVs, for vaccine development. Using two Streptococcus pneumoniae proteins as antigens, it was shown that mucosally administered Salmonella OMVs displaying high levels of antigens at the surface induced strong protection in a murine model of streptococcal colonization, without the need for a mucosal adjuvant. Importantly, reduction in bacterial recovery from the nasal cavity was correlated with local production of antigen-specific IL-17A. Furthermore, the protective efficacy and the production of antigen-specific IL-17A, and local and systemic IgGs, were all improved at increased concentrations of the displayed antigen.

### Materials and Methods

Bacterial Strains and Growth Conditions:

S. Typhimurium SL3261 ΔtoIRA [21], and E. coli TOP10F′ and BL21(DE3) were grown at 37° C. in LB medium containing 0.2% glucose. When appropriate, kanamycin was used at a concentration of 25 μg/ml and chloramphenicol at 30 μg/ml. S. pneumoniae TIGR4 (Tettelin et al (2001) Science 293(5529):498-506) was grown and vaccination stocks containing 10⁶ colony forming units (CFU)/10 μl in phosphate buffered saline (PBS) were prepared as described (Cron et al (2011) Infect Immun 79(9):3697-710).

Plasmid Construction:

All reagents were purchased from Roche, except for Phusion DNA polymerase (Finnzymes) and SacI (New England Biolabs).

All plasmids used for expression of Hbp (derivatives) have a pEH3 backbone (Hashemzadeh-Bonehi et al (1998) Mol Microbiol 30(3):676-8). Plasmids pHbpD(Δd1), pHbpD(Δd2) and pHbpD(d4in), in which sequences coding for side domains d1, d2 and d4 of the Hbp passenger were substituted for Gly/Ser-encoding linkers containing SacI and BamHI restriction sites have been described (Jong et al (2012), supra).

Fragments of pspA encoding the N-terminal and the C-terminal portions of the α-helical domain, the LFB domain and the Pro-rich region, and of ply encoding an N-terminal (F1; aa 1-156), a central (F2; aa 145-421) and a C-terminal fragment (F3; aa 357-471) were amplified with flanking SacI/BamHI sites using chromosomal DNA of S. pneumoniae TIGR4 as template (GenBank accession no. AE005672) and the primers listed in Table 2. The resulting PCR amplicons were digested with SacI and BamHI and inserted into the hbp orfs of plasmids pHbpD(Δd1), pHbpD(Δd2) or pHbpD(d4in), which had been digested with the same restriction enzymes. This approach resulted in plasmids pHbpD(Δd1)-PspA(α1), pHbpD(Δd1)-PspA(LFBD), pHbpD(Δd1)-Ply(F1), pHbpD(Δd2)-PspA(α2), pHbpD(Δd2)-Ply(F3) and pHbpD(Δd4)-PspA(PRR).

To create a plasmid for expression of Hbp fused to both the α1 and the α2 fragments of PspA, the NdeI/NsiI fragment of pHbpD(Δd1)-PspA(α1) was substituted for that of pHbpD(Δd2)-PspA(α2), resulting in pHbpD-PspA(α1α2). Similarly, the NdeI/NsiI fragment of pHbpD(Δd1)-Ply(F1) was substituted for that of pHbpD(Δd2)-Ply(F3), yielding plasmid pHbpD-Ply(F1F3) for expression of a chimera containing both fragments F1 and F3 of pneumolysin. Finally, a plasmid was created for expression of Hbp fused to the LFBD and PRR fragments of PspA by replacing the NsiI/KpnI fragment of pHbpD(Δd1)-PspA(LFBD) for that of pHbpD(Δd4)-PspA(PRR).

TABLE 2
Primer sequences

| Name | Sequence (5′ → 3′) | SEQ ID NO |
|---|---|---|
| PspA(PRR)-fw | cggggagctccgagttaggccctgatggag | 23 |
| PspA(PRR)-rv | tgccggatccttgtttccagcctgtttttgg | 24 |
| PspA(LFBD)-fw | cggggagctcccttgctggtgcagatcctgatgatg | 25 |
| PspA(LFBD)-rv | tgccggatccagtttcttcttcatctccatcag | 26 |
| PspA(α2) fw | cggggagctccgtaagagcagttgtagttcc | 27 |
| PspA(α2) rv | tgccggatcctgtgccatcatcaggatctgcaccagc | 28 |
| PspA(α1) fw | cggggagctccgaagaatctccacaagttgtc | 29 |
| PspA(α1) rv | tgccggatccatttggttcaggaactacaactg | 30 |
| Ply(F1)-fw | cggggagctccatggcaaataaagcagtaaatgac | 31 |
| Ply(F1)-rv | tgccggatccgtgagccgtgattttttcatac | 32 |
| Ply(F3)-fw | cggggagctccgcttacagaaacggagatttactg | 33 |
| Ply(F3)-rv | tgccggatccgtcattttctaccttatcttctac | 34 |
| nHis-pspA(dN31)-fw | aaaccatgggccatcatcatcatcatcatcatcacag | 35 |
| | cagcggcgaagaatctccacaagttgtcg | |
| pspA-rv | tttcatatgttaaacccattcaccattgg | 36 |

Constructs for expression and purification of N-terminally His-tagged PspA and PdT were created using the pET16b(+) vector (Novagen). For cloning of pspA, a fragment corresponding to aa 31 to 744 was amplified PCR using S. pneumoniae TIGR4 chromosomal DNA as template. The forward primer nHis-pspA(dN31)-fw was designed to contain a His₈-epitope encoding sequence and an NcoI restriction site, while the reverse primer pspA-rv contained an NdeI site. A synthetic E. coli-codon-optimized DNA fragment encoding PdT flanked with NcoI and NdeI restriction sites was ordered from Life Technologies. The fragments were digested with NcoI and NdeI and ligated into pET16b(+), digested with the same enzymes, resulting in pET16b(+)::PspA(31-744) and pET16b(+)::PdT. The nucleotide sequences of all constructs were confirmed by DNA sequencing.

Hbp Expression, OMV Isolation and Protein Analysis:

S. Typhimurium SL3261 ΔtoIRA harboring pEH3 vectors was grown until an OD₆₆₀ of ˜0.6, at which expression of Hbp derivatives was induced from the lacUV5 promoter in the presence of 1 or 100 μM IPTG for 1 hour.

To isolate OMVs, culture supernatants obtained by low-speed centrifugation were passed through 0.45-μm-pore-size filters (Millipore) and centrifuged at 208 000 g for 60 min, separating OMVs from soluble proteins. Pelleted OMVs were washed by re-suspension in PBS containing 500 mM NaCl (1 OD unit of OMVs per μl) and centrifugation at 440 000 g for 2 h, after which they were taken up in PBS containing 15% glycerol (1 OD unit of OMVs per μl). An amount of 1 OD unit of OMVs is derived from 1 OD₆₆₀ unit of cells.

Proteinase K accessibility of OMV proteins was analyzed as described (Daleke-Schermerhorn et al (2014), supra), and proteins were analyzed by SDS-PAGE and Coomassie G-250 (BioRad) staining or immunoblotting using antisera recognizing the β-domain of Hbp (SN477), PspA or pneumolysoid, the detoxified derivative of Ply (own lab collection). Densitometric analysis on Coomassie-stained gels was carried out using a Molecular Imager GS-800 Calibrated Densitometer (Biorad) and Quantity One software (Biorad).

Expression and Purification of his-Tagged PspA and PdT:

Overnight cultures of E. coli BL21(DE3) harboring pET16b(+) plasmids for expression of PspA and PdT were grown at 37° C. in LB medium supplemented with ampicillin (100 μg/ml) and glucose (0.4%). The next morning, cultures were diluted to an OD₆₆₀ of 0.05 in fresh medium and growth was continued under the same conditions. When the cultures had reached an OD₆₆₀ of 0.6, recombinant protein production was induced by the addition of 100 μM IPTG for 2 h. Cells were harvested by low-speed centrifugation, re-suspended in ice cold PBS (pH 7.4) containing 5 mM imidazole and 300 mM NaCl, and lysed in a One Shot cell disrupter (Constant Systems Ltd) at 1.72 kbar. Unbroken cells were removed by low-speed centrifugation. Thereafter, soluble proteins were obtained by high-speed centrifugation at 208 000 g at 4° C. for 90 min. For PdT, His-tagged protein present in the high-speed supernatant was purified by affinity chromatography using HiTrap TALON crude columns (GE Healthcare). Bound PdT was eluted over a gradient of 5-500 mM imidazole in PBS (pH 7.4) containing 300 mM NaCl. Fractions containing His-tagged PdT were pooled and dialyzed against PBS containing 15% glycerol. The same procedure was followed to isolate His-tagged PspA, except that the high-speed supernatant and the buffers used during HiTrap TALON affinity chromatography contained 8 M urea.

Mouse Immunizations and Challenge:

Seven week-old female C57BL/6 mice (Charles River Laboratories) were intranasally (i.n.) immunized three, two or one time(s) with 8 OD units of OMVs (corresponding to ˜4 μg total protein) in a volume of 10 μl, at two-week intervals, under anesthesia (2.5% v/v isoflurane, AU Veterinary Services). Three weeks after the final immunization, mice were challenged i.n. with 106 CFU of S. pneumoniae TIGR4 (Cron et al (2011), supra). Three days after infection, mice were euthanized, and blood and mucosal nasal tissue were harvested. Nasal tissue was homogenized using an IKA T10 basic blender, and serially diluted samples were plated on Gentamicin Blood Agar (Mediaproducts BV) to determine bacterial recovery (log CFU/organ). All animal work was performed with approval of the Radboud University Medical Center Committee for Animal Ethics. Consequently, three mice were euthanized and excluded from further experimental analysis after reaching a humane endpoint, i.e. over 20% weight loss, potentially caused by a reaction to lipopolysaccharide present in the OMVs.

Detection of Antibody Responses by Enzyme-Linked Immunosorbant Assay Analysis:

Maxisorp high binding affinity plates (Nunc) were coated with 2 μg/ml purified PspA or PdT in carbonate coating buffer (0.1 M carbonate/bicarbonate pH 9.6) at 4° C. overnight. The next day, wells were blocked with 1% BSA (Sigma) and subsequently incubated for 1 h at 37° C. with serum or nasal samples from individual mice. Thereafter, the wells were incubated with primary anti-mouse IgG-alkaline phosphatase (Sigma) or primary anti-mouse IgA-alkaline phosphatase (Southern Biotech) for 1 h at 37° C. Between and after the incubations steps, all wells were washed with PBS containing 0.05% Tween-20 (Merck). Samples were developed using 1 mg/ml p-nitrophenylposphate in substrate buffer (1 M diethanolamine, 0.5 mM MgCl₂ pH 9.8) (Calbiochem, VWR) and the optical density was measured at 405 nm 10 and 30 minutes after substrate addition.

Measurement of Local IFNγ and IL-17A:

Cytokine production in mouse nasal samples were determined with Cytometric Bead Array (Becton Dickinson) according to manufacturer's instructions, using the Mouse Enhanced Sensitivity buffer kit in combination with the Enhanced Sensitivity Flex set for IFNγ and IL-17A (Becton Dickinson). Concentrations were calculated using Soft Flow FCAP Array v1.0 (Becton Dickinson).

Statistical Analyses:

All statistical analyses were performed using GraphPad Prism version 5.0 (Graphpad Software). For bacterial recovery data, the Grubbs outlier test was used to test for significant outliers. The One-Way Anova Kruskall Wallis with Bonferroni post-test for multiple groups or Mann-Whitney t test for two groups were used for comparisons of protection and immune responses. To determine the relation between IL-17A and protection, a Spearman Correlation test was applied.

### Results

Selection and Fusion of PspA and Pneumolysin Fragments to Hbp:

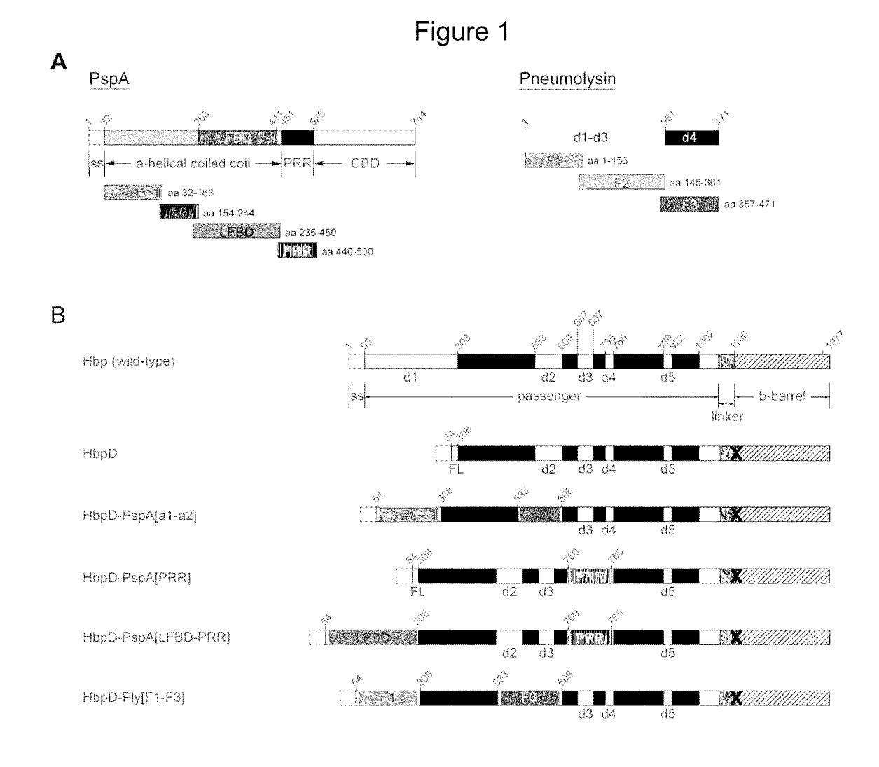

To facilitate efficient expression of the model antigens PspA and Ply via the Hbp display system, these two complex, multi-domain proteins were split into shorter fragments (FIG. 1A). To avoid potential destruction of immune epitopes, fragments were designed to partially overlap with adjacent sequences. PspA was divided into four fragments that cover the N-terminal, surface-exposed part of the protein (FIG. 1A). Of these, two fragments derived from the N-terminal portion of α-helical coiled coil domain (“α1” and “α2”) were fused to a single Hbp carrier, yielding HbpD-PspA[α1-α2] (SEQ ID NO:4; FIG. 1B). A second construct, HbpD-PspA[LFBD-PRR] (SEQ ID NO:6; FIG. 1B), was created by combining Hbp with two fragments corresponding to the lactoferrin-binding domain (‘LFBD’) and the conserved Pro-rich region (‘PRR’) of PspA, respectively. Similarly, pneumolysin was divided into three fragments (FIG. 1A), of which an N-terminal (′F1′) and a C-terminal fragment (‘F3’) were fused to a single Hbp carrier to create HbpD-Ply[F1-F3] (SEQ ID NO:7; FIG. 1B). Unfortunately, the central pneumolysin fragment (‘F2’; FIG. 1A), that includes the membrane-penetrating D3 domain, caused lysis upon expression in the context of Hbp and was therefore excluded from further studies.

Efficient Display of PspA and Pneumolysin Fragments at the Surface of Salmonella OMVs:

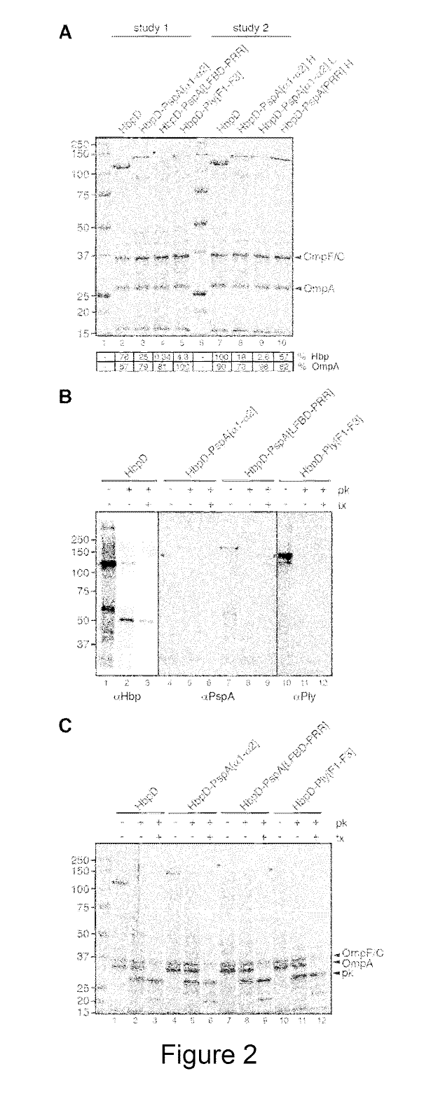

The three Hbp chimeras were expressed under control of a lacUV5 promoter in a previously described ΔtoIRA derivative of the attenuated Salmonella Typhimurium strain SL3261 that produces large amounts of OMVs (Daleke-Schermerhorn et al (2014), supra). A display derivative of Hbp lacking domain d1, HbpD (SEQ ID NO:3; FIG. 1B; Jong et al (2012), supra), was produced in the same strain and the resulting vesicles served as a negative control in subsequent studies. OMVs were isolated from cell-free culture supernatants by ultracentrifugation, after which they were washed with PBS containing a high concentration of NaCl to remove peripherally attached soluble contaminants. The absence of live bacteria was verified by plating, and OMVs were confirmed to contain the Hbp-antigen chimera by SDS-PAGE (FIG. 2A) and immunoblotting (FIG. 2B). Importantly, and similar to the HbpD control, all three chimeras were exposed at the OMV surface as judged by their sensitivity to externally added Proteinase K (FIGS. 2B and C). In contrast, degradation of the protease-sensitive C-terminal periplasmic domain of OmpA occurred only after permeabilization of the OMVs with Triton X-100, confirming the integrity and membrane orientation of the vesicles (FIG. 2C). Differences in expression levels of the various HbpD-antigen fusion proteins are likely determined by the difference in complexity of the inserted fragments. Nevertheless, all Hbp-antigen fusions were visible after Coomassie staining indicating that the expression levels are substantial.

Salmonella OMVs Displaying PspA Fragments Protect Against Pneumococcal Colonization:

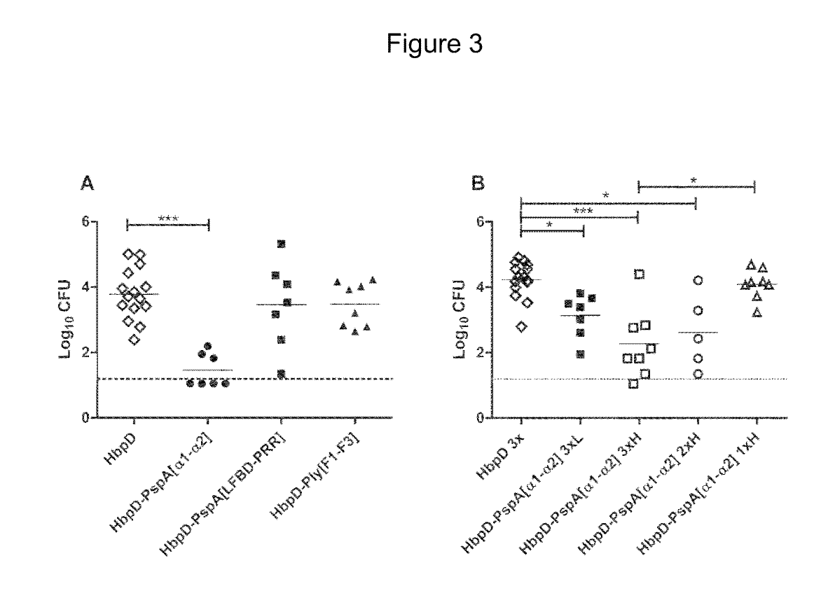

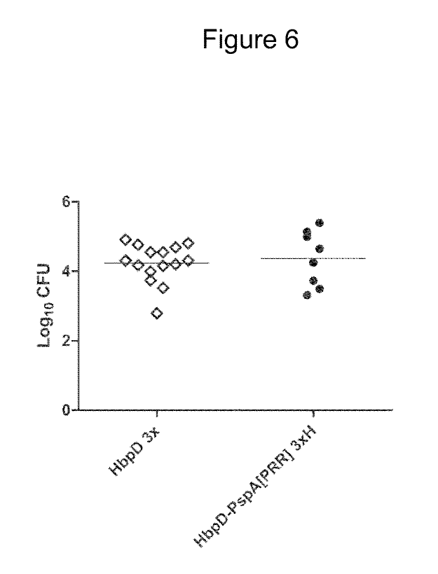

To investigate whether Salmonella OMVs displaying PspA and Ply fragments at the surface can confer protection against pneumococcal colonization, we made use of a previously established mouse model (Wu et al (1997) Microb Pathog 23(3):127-37). Mice were intranasally immunized three times with two-week intervals, and three weeks after the final immunization, they were intranasally inoculated with the S. pneumoniae serotype 4 TIGR4 strain. Recovery of live pneumococci from nasal tissue three days post-infection revealed that mice immunized with OMVs displaying HbpD-PspA[α1-α2] were significantly protected compared to mice that received OMVs displaying HbpD (FIG. 3A). Remarkably, over 50% of the mice that received OMVs/HbpD-PspA[α1-α2] completely cleared the pneumococci within three days post-infection. In contrast, mice immunized with OMVs displaying either HbpD-PspA[LFBD-PRR] or HbpD-Ply[F1-F3] showed little, and in the case of HbpD-PspA[LFBD-PRR] rather variable protection, which did not significantly differ from the negative control. To investigate whether the reduced protection may be due to insufficient expression levels of HbpD-PspA[LFBD-PRR] and HbpD-Ply[F1-F3] (FIG. 2A), immunization was repeated with OMVs displaying one of the antigen fragments, i.e. PspA[PRR] (FIG. 2A, lane 10; SEQ ID NO:5), at even ˜3-fold higher levels than PspA[α1-α2] (FIG. 2A, lane 8). However, no reduction in bacterial load was observed (FIG. 6). Together these data show that the OMV/Hbp platform is suitable for intranasal antigen delivery. Furthermore, the N-terminal half of the α-helical coiled coil domain of PspA is able to induce protection against pneumococcal colonization.

Immunogenicity of Salmonella OMVs Displaying Pneumococcal Antigens:

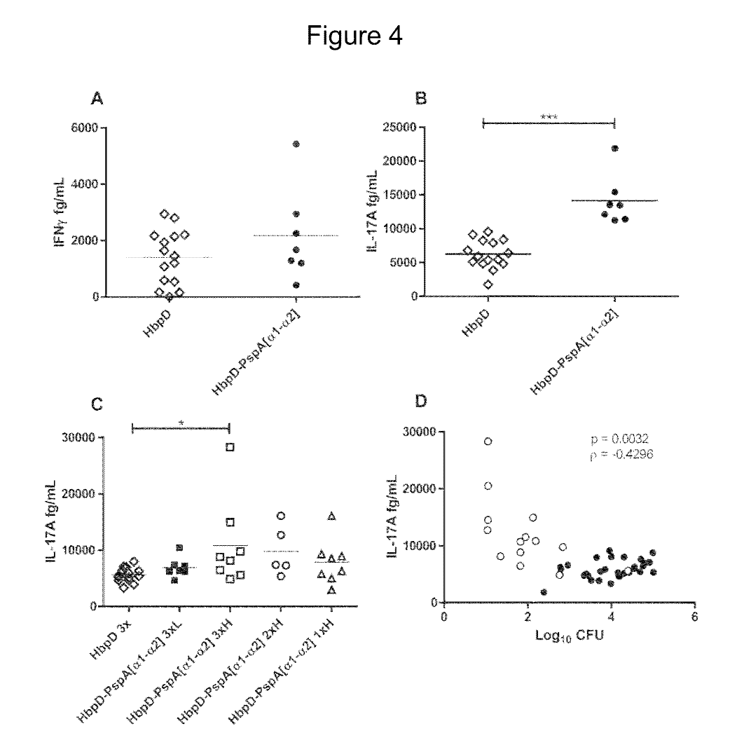

To shed light on the underlying mechanism behind the observed protection, we quantified the levels of IFNγ and IL-17A in nasal tissue of mice immunized with OMVs displaying HbpD-PspA[α1-α2] or the control protein HbpD. Of note, because the vaccinated mice were subjected to subsequent pneumococcal infection, the cytokine levels measured at three days post-challenge reflect recall responses of immunization-induced memory towards the pneumococcal antigen fragments. Interestingly, while both groups produced similar levels of IFNγ (FIG. 4A), mice immunized with OMVs/HbpD-PspA[α1-α2] produced significantly higher levels of IL-17A levels compared to control mice (FIG. 4B). This strongly suggests that local production of IL-17A, but not IFNγ, is important for protection against pneumococcal colonization.

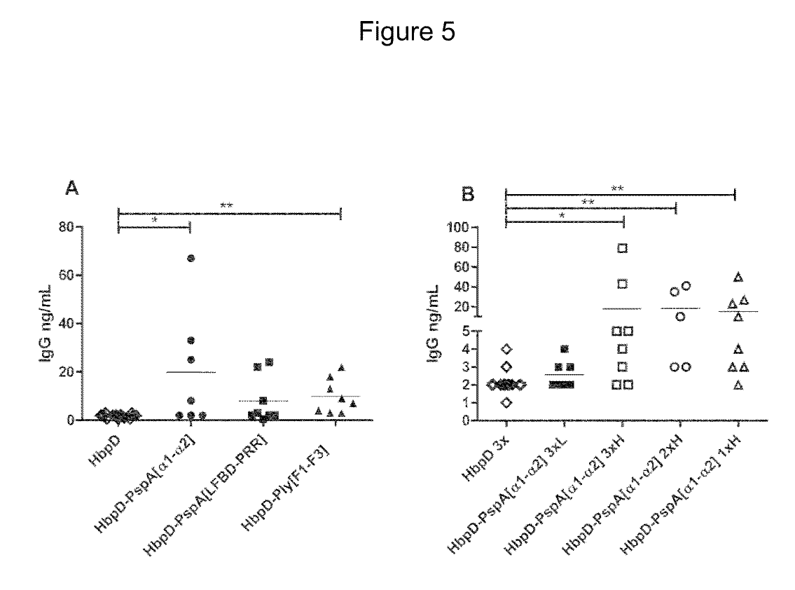

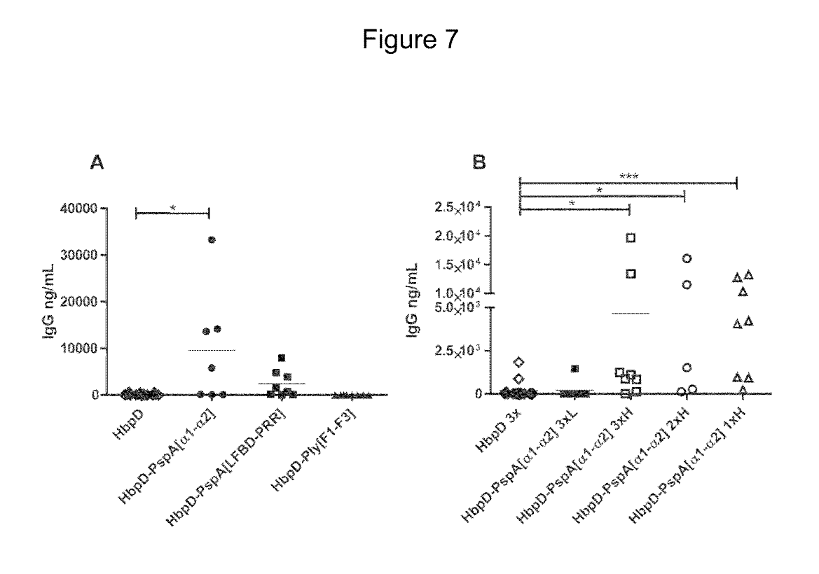

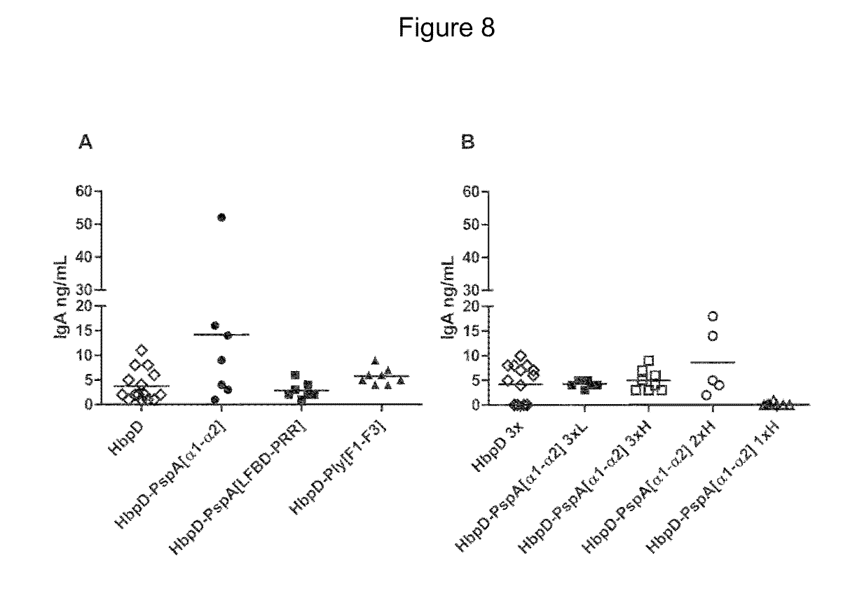

Because IgGs are important for bacterial opsonization and initiation of robust immune responses, we determined the levels of PspA- and Ply-specific IgGs and IgAs in nasal tissue homogenates and sera of immunized mice (FIGS. 5A, 7A and 8A). Interestingly, significant yet variable local and systemic antigen-specific IgG production was detected upon immunization with OMVs/HbpD-PspA[α1-α2]. In contrast, with the exception of apparently lower local IgGs induced by OMVs/HbpD-Ply[F1-F3], no antibody responses were detected in the other groups. Moreover, there were no differences observed for local IgA production between the treatment groups.

Together these results show that the OMV/Hbp platform induces local antigen-specific IL-17A responses. Additionally, the platform can induce local and systemic humoral responses.

Level of Protection is Influenced by the Vaccine-Associated Antigen Load and the Number of Immunizations:

Although intrinsic properties of the α1/α2 fragments of PspA are apparently crucial for protection, the strikingly high expression levels of HbpD-PspA[α1-α2] (FIG. 2A) prompted us to investigate whether antigen levels represent an important determinant for immune responses and protection. To this end, mice were immunized with two new batches of OMVs displaying HbpD-PspA[α1-α2] at high levels similar to the first batch or at approximately ˜7-fold lower levels (FIG. 2A). Similar to the previous experiment, mice immunized with OMVs expressing high levels of HbpD-PspA[α1-α2] produced significantly elevated levels of antigen-specific IL-17A in nasal tissue (FIG. 4C), and local and systemic antigen-specific IgG, but not local antigen-specific IgA (FIGS. 5B, 7B and 8B). In contrast, IL-17A and IgG levels were low or undetectable in samples from mice immunized with OMVs displaying low levels of HbpD-PspA[α1-α2], and did not significantly differ from those of control mice that received three doses of OMVs without pneumococcal protein fragments (FIGS. 4C, 5B and 7B). Furthermore, although low PspA[α1-α2] induced some protection against pneumococcal colonization, a clear trend suggested that enhanced expression of PspA[α1-α2] improved protection (FIG. 3B).

To investigate whether similar levels of protection could be reached with fewer immunizations, two additional groups of mice were included that received one or two immunizations with OMVs expressing high levels of HbpD-PspA[α1-α2]. Antibody analysis revealed significantly elevated local and systemic IgG, but not local IgA levels independently of the number of immunizations (FIGS. 5B, 7B and 8B). However, although slightly more IL-17A was detected after two vaccinations, three immunizations were required to induce significantly higher IL-17A production compared to control mice (FIG. 4C). Moreover, three immunizations offered significantly better protection than two immunizations, while one immunization was not sufficient to induce protection as compared with the control (FIG. 3B).

In conclusion, the antigen abundance and the number of immunizations directly influence the levels of nasal IL-17A and protection against S. pneumoniae colonization. Importantly, production of nasopharyngeal IL-17A significantly correlated with reduced bacterial recovery from the nasal tissue (p=0.0032) (FIG. 4D), suggesting that local IL-17A responses are crucial for protective memory responses induced by our OMV/Hbp antigen display platform.

What is claimed is:

1. A fusion protein, comprising:
i. a passenger domain comprising a beta stem domain from an autotransporter protein, wherein the beta stem forming sequence of the passenger domain is essentially intact;
ii. a translocator domain from an autotransporter protein;
iii. a signal peptide that targets the fusion protein to the inner membrane of a Gram negative bacterium; and
iv. at least one antigenic fragment;
wherein the passenger domain of the autotransporter in its native form comprises at least one side domain, and wherein said antigenic fragment replaces or partly replaces said side domain; and
wherein said at least one antigenic fragment is a fragment of a protein from a bacterium of the genus Streptococcus.

2. The fusion protein according to claim 1, wherein said at least one antigenic fragment is a fragment of a protein from Streptococcus pneumoniae.

3. The fusion protein according to claim 2, wherein said at least one antigenic fragment is a fragment of pneumococcal surface protein A from Streptococcus pneumoniae.

4. The fusion protein according to claim 3, wherein said at least one antigenic fragment is a fragment of the α-helical coiled-coil domain of pneumococcal surface protein A from Streptococcus pneumoniae.

5. The fusion protein according to claim 4, wherein said α-helical coiled-coil domain comprises an amino acid sequence selected from the group consisting of SEQ ID NO: 9 and sequences having at least 30% identity thereto.

6. The fusion protein according to claim 1, wherein said at least one antigenic fragment consists of 20-250 amino acids.

7. The fusion protein according to claim 1, comprising at least a first antigenic fragment α1 and a second antigenic fragment α2 which are different, overlapping or non-overlapping, fragments of said α-helical coiled-coil domain of pneumococcal surface protein A from Streptococcus pneumoniae, wherein the passenger domain of the autotransporter in its native form comprises at least two side domains, and wherein each of said antigenic fragments α1 and α2 replaces or partly replaces a separate side domain.

8. The fusion protein according to claim 7, wherein the amino acid sequence of α1 consists of 100-150 amino acid residues, for example from 120-140 amino acid residues, for example 125-135 amino acid residues.

9. The fusion protein according to claim 7, wherein the amino acid sequence of α1 comprises a sequence selected from the group consisting of SEQ ID NO: 10 and sequences having at least 30% identity thereto.

10. The fusion protein according to claim 9, wherein the amino acid sequence of α1 comprises SEQ ID NO: 10.

11. The fusion protein according to claim 9, wherein the amino acid sequence of α1 consists of a sequence selected from the group consisting of SEQ ID NO: 10 and sequences having at least 30% identity thereto.

12. The fusion protein according to claim 11, wherein the amino acid sequence of α1 consists of SEQ ID NO: 10.

13. The fusion protein according to claim 7, wherein the amino acid sequence of α2 consists of 60-110 amino acid residues.

14. The fusion protein according to claim 7, wherein the amino acid sequence of α2 comprises a sequence selected from the group consisting of SEQ ID NO: 11 and sequences having at least 30% identity thereto.

15. The fusion protein according to claim 14, wherein the amino acid sequence of α2 comprises SEQ ID NO: 11.

16. The fusion protein according to claim 14, wherein the amino acid sequence of α2 consists of a sequence selected from the group consisting of SEQ ID NO: 11 and sequences having at least 30% identity thereto.

17. The fusion protein according to claim 16, wherein the amino acid sequence of α2 consists of SEQ ID NO: 11.

18. The fusion protein according to claim 1, wherein the passenger domain (i) and the translocator domain (ii) are derived from a serine protease autotransporter of Enterobacteriaceae (SPATE) protein.

19. The fusion protein according to claim 18, wherein the SPATE protein is selected from the group consisting of hemoglobin-binding protease (Hbp), extracellular serine protease (EspC) and temperature-sensitive hemagglutinin (Tsh) from Escherichia coli.

20. The fusion protein according to claim 19, wherein the SPATE protein comprises an amino acid sequence selected from the group consisting of SEQ ID NO: 1, SEQ ID NO: 2 and homologous sequences having at least 35% identity thereto.

21. The fusion protein according to claim 20, wherein said passenger domain in its native form comprises five side domains, which are defined by amino acids 54-308, 533-608, 657-697, 735-766 and 898-922 of SEQ ID NO: 1 or SEQ ID NO: 2.

22. The fusion protein according to claim 21, wherein one of said α1 and α2, when present, is inserted into, replaces or partly replaces the side domain defined by amino acids 54-308, and the other one of said α1 and α2, when present, is inserted into, replaces or partly replaces the side domain defined by amino acids 533-608.

23. The fusion protein according to claim 1, which does not comprise a cleavage site, or comprises a disrupted cleavage site, such that the fusion protein is displayed on the surface of a cell in which it is expressed.

24. A polynucleotide encoding a fusion protein according to claim 1.

25. An expression vector comprising a polynucleotide according to claim 24.

26. A gram-negative bacterial host cell comprising an expression vector according to claim 25.

27. The host cell according to claim 26, which belongs to the family Enterobacteriaceae.

28. The host cell according to claim 27, which belongs to the species Salmonella enterica.

29. The host cell according to claim 28, which is a ΔtoIRA derivative of S. Typhimurium strain SL3261.

30. A method of expressing a fusion protein comprising the steps of
i. providing a host cell according to claim 26; and
ii. culturing said host cell under conditions suitable for expression of said fusion protein.

31. A method of producing outer membrane vesicles displaying a fusion protein on their surface, comprising:
expressing a fusion protein using the method according to claim 30; and
shedding of vesicles from the outer membrane of the host cell to obtain outer membrane vesicles displaying the fusion protein on their surface.

32. An outer membrane vesicle, displaying at least one fusion protein according to claim 1 on its surface.

33. A vaccine comprising an outer membrane vesicle according to claim 32.

34. A method of inducing protective immunity against Streptococcus, comprising the step of administering a vaccine according to claim 33 to a subject in need thereof.

35. The method according to claim 34, wherein said protective immunity comprises protection against streptococcal colonization.

36. The method according to claim 34, wherein said protective immunity is characterized by a high expression of IL-17A.

37. The method according to claim 36, wherein said expression of IL-17A is localized in nasopharyngeal tissue.

38. The method according to claim 34, wherein said vaccine is mucosally administered.

39. The method according to claim 38, wherein said vaccine is intranasally administered.

40. The method according to claim 34, which induces protective immunity against a streptococcal disease.

41. The method according to claim 34, wherein said protective immunity comprises protection against colonization by Streptococcus pneumoniae.

42. The method according to claim 41, which induces protective immunity against a pneumococcal disease selected from the group consisting of pneumonia, meningitis, otitis media, bacteremia, sepsis and acute exacerbations of chronic bronchitis, sinusitis, arthritis and conjunctivitis.

43. The fusion protein according to claim 7, wherein the amino acid sequence of α2 consists of 70-100 amino acid residues.

44. The fusion protein according to claim 7, wherein the amino acid sequence of α2 consists of 75-95 amino acid residues.

45. The host cell according to claim 27, which is selected from the group consisting of Escherichia coli, Salmonella spp., Vibrio spp., Shigella spp., Pseudomonas spp., Burkholderia spp. and Bordetella spp.

46. The host cell according to claim 45, which belongs to the subspecies Salmonella enterica subsp enterica.

47. The host cell according to claim 46, which belongs to serovar Typhimurium.

48. The host cell according to claim 47, which is strain SL3261.

49. The method according to claim 40, wherein the streptococcal disease is selected from the group consisting of pneumonia, endocarditis, meningitis, otitis media, bacteremia, sepsis, pharyngitis, respiratory infections, dental caries and acute exacerbations of chronic bronchitis, sinusitis, arthritis and conjunctivitis.
